(19) United States
(12) Patent Application Publication
(10) Pub. No.: US 2025/0018125 A1
(43) Pub. Date: Jan. 16, 2025

(54) SYSTEM AND METHOD FOR A CONSTANT FORCE SYRINGE PUMP ACCOMODATING SYRINGES OF DIFFERENT SIZES

(71) Applicant: KORU Medical Systems, Inc., Mahwah, NJ (US)

(72) Inventors: BRIAN CHRISTOPHER CASE, Lake Villa, IL (US); ERIK MARIUS KARLSEN, Vernon, NJ (US); STEVEN ANDREW WATTS, San Diego, CA (US); PETER ANDREW SMITH, Zebulon, NC (US); GABRIEL DORAN JEFFER, Durham, NC (US); DEREK TEIG ROBERTS, Chicago, IL (US); THOMAS RALPH BLACKBURN, III, Holly Springs, NC (US)

(73) Assignee: KORU Medical Systems, Inc., Mahwah, NJ (US)

(21) Appl. No.: 18/767,796

(22) Filed: Jul. 9, 2024

Related U.S. Application Data
(60) Provisional application No. 63/526,050, filed on Jul. 11, 2023.
(60) Provisional application No. 63/603,176, filed on Nov. 28, 2023.

(51) Int. Cl. A61M 5/315

(52) U.S. Cl. CPC A61M 5/31565

(57) ABSTRACT

Provided is a constant force pump accommodating syringes of different sizes. The system is provided by an enclosure having a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; a sizer structured and arranged to determine the size of the chamber; an adjustable driver structured and arranged to provide a constant force between the pusher and the puller, the constant force adjustably selected by the size of the chamber. An associated method of use is provided as well.

### CROSS REFERENCE TO RELATED APPLICATIONS

This application claims the benefit under 35 U.S.C. § 119 (e) of U.S. Provisional Application No. 63/526,050 filed Jul. 11, 2023, and entitled SYSTEM AND METHOD FOR A CONSTANT FORCE SYRINGE PUMP ACCOMMODATING SYRINGES OF DIFFERENT SIZES, the disclosure of which is incorporated herein by reference. This application also incorporates by reference U.S. Provisional Application No. 63/603,176 filed Nov. 28, 2023, entitled SYSTEM AND METHOD FOR A CONSTANT FORCE SYRINGE PUMP ACCOMMODATING SYRINGES OF DIFFERENT SIZES.

### FIELD OF THE INVENTION

The present invention relates generally to mechanical syringe pumps for delivering a liquid medicant to a patient, and more specifically to a mechanical constant force syringe pump advantageously adapting to the different size of each syringe to ensure the proper constant force is applied for dispensation of the liquid medicant to the patient in accordance with the prescribed rate of medicant flow for the presently engaged syringe.

### BACKGROUND

Infusion systems for the delivery of liquid pharmaceuticals are widely used and relied upon by patients and care givers alike to provide infusion therapy.

One form of infusion therapy is Immune Globulin (Ig) therapy, and it is frequently used to improve the quality of life for patients with conditions such as Primary Immune Deficiency (PID), Secondary Immune Deficiency (SID), Chronic Inflammatory Demyelinating Polyradiculoneuropathy (CIPD), and Severe Combined Immunodeficiency (SCID). Historically, Ig therapy has been administered intravenously (IVIg) every 3-4 weeks, which can result in inconsistent serum levels and burdensome infusion experiences for patients.

Subcutaneous Ig therapy (SCIg) is a more convenient option that allows for consistent serum levels and can be done at home. Subcutaneous administrations have been shown to maintain a more constant IgG blood level which provides the patient with a consistent quality of life, and less breakthrough infections. Additionally, infusion to subcutaneous tissues is often preferred over intravenous delivery which is often accompanied by flu-like symptoms following such intravenous delivery. However, SCIg has its own challenges, such as the need for patients to prepare and administer the infusions themselves.

At least one challenge relates to the pump system used for subcutaneous Ig therapy. There are essentially two varieties of home pump systems—constant pressure and constant flow. With a constant flow rate system, the pressure is increased in response to any flow restriction no matter if such a restriction is the build-up of pressure in the patient's tissues or an element of the delivery system. This can result in an administration of the liquid at an unsafe pressure. As such, the patient may suffer a wide range of symptoms, including, but not limited to, anaphylaxis, overdose, histamine reactions, morbidity, and mortality.

In contrast, a constant pressure pump generates a safe and limited constant pressure. If there is a pinch in the tubing blockage in the infusion system or blockage in the patient's body (such as by saturation of the tissues), such a blockage results in resistance to the flow and affects the flow rate, not the pressure, i.e., the flow rate decreases as the pressure increases. As such, constant pressure pump systems have been found to be safer and are often more financially acceptable to users.

But there is also the issue of dosage. Frequently dosage is pre-determined and provided in pre-filled syringes that may be disposed within a constant pressure pump. However, each person is different in size, shape, and tissue makeup among many factors. Bodyweight itself could necessitate a prescription for a 17 g (85 ml) dose for Ig therapy for one person, but only 10 g for another person. However, syringes come in a variety of sizes as well—such as 5, 10, 20 and 50 ml, with each varying in length as well as diameter.

If the medication is provided in a syringe other than the one the constant pressure pump is designed for, the patient or caregiver must transfer the liquid medicant from the pre-filled syringe into a new and sterile pump compliant syringe. Such a transfer process is not without issues and introduces the opportunity for problems, such as but not limited to complete transfer, contamination, and general stress and concern.

As 10 ml syringe has a different length and diameter from that of a 50 ml syringe, a different amount of force should be applied to the syringe to generate the needed constant pressure to provide the same expected and intended initial flow rate while also providing the safeguard of the flow rate from the pump decreasing as resistance pressure increases due to the infusion system itself or issues within the patient.

More simply stated, if a 10 ml syringe was disposed in a pump designed for a 50 ml syringe, the constant force applied to the 10 ml syringe would be far greater than appropriate. Likewise, a 50 ml syringe disposed in a pump intended for a 10 ml syringe, the applied constant force would be too little and the intended flow would not be provided.

Indeed, U.S. Pat. No. 10,376,636 entitled Compact Mechanical Pump specifically teaches that each embodiment of the Compact Mechanical Pump as disclosed is intended for a syringe of a specific size, and “if the user attempts to use a 30 ml syringe in a pump suited for a 20 ml syringe, the rejection ramps will eject the syringe from the base upon attempted closure of the cover”

Hence there is a need for a method and system that is capable of overcoming one or more of the above identified challenges.

### SUMMARY OF THE INVENTION

Our invention solves the problems of the prior art by providing novel systems and methods for a mechanical constant force pump assembly as a constant force syringe pump accommodating syringes of different sizes.

In particular, and by way of example only, according to one embodiment of the present invention, provided is a constant force syringe pump assembly accommodating syringes of different sizes including: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; a sizer structured and arranged to determine the size of the chamber; an adjustable driver structured and arranged to provide a constant force between the pusher and the puller, the constant force adjustably selected by the size of the chamber; whereby slidably moving the puller distally when the syringe is seated in the base causes the pusher to contact and exert a substantially constant force specific for the size of the chamber on the head of the plunger, the constant force sufficient to move the plunger of the syringe; and a cover connected to the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

In yet another embodiment, provided is a constant force syringe pump assembly accommodating syringes of different sizes including: an expandable base having a proximal end and a distal end, the base including: a first base section and a second base section, wherein the first base section is in sliding engagement with the second base section such that the first base section and the second base section are slidably movable relative to each other between a compacted position and an expanded position, wherein the base in the expanded position is adapted to seat a syringe having a plunger slidably disposed within a chamber having an outlet, the chamber having a length and a diameter, the plunger having a head; a pusher in sliding engagement with the base, wherein the pusher is dimensioned to contact the head of the plunger; a puller in sliding engagement with the base; a sizer structured and arranged to determine the size of the chamber; an adjustable driver structured and arranged to provide a constant force between the pusher and the puller, the constant force adjustably selected by the size of the chamber; whereby slidably moving the puller distally when the syringe is seated in the base causes the pusher to contact and exert a substantially constant force specific for the size of the chamber on the head of the plunger, the constant force sufficient to move the plunger of the syringe; and an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

Yet for another embodiment, provided is a constant force syringe pump assembly accommodating syringes of different sizes including: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; a sizer structured and arranged to determine the size of the chamber; a plurality of different springs, each spring having a first end and a second end, the first end of each spring connected to the pusher, the second end of each spring selectively attached to the puller, the selection of at least one selectively attached spring from the plurality of springs based on the size of the chamber; whereby slidably moving the puller distally when the syringe is seated in the base causes the pusher to contact and exert a substantially constant force specific for the size of the chamber on the head of the plunger, the constant force sufficient to move the plunger of the syringe; and a cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one selectively attached spring; wherein the base and cover enclose the chamber body, the at least one selectively attached spring, puller and pusher are structured and arranged to provide the constant force specific to the size of the chamber to the plunger of the syringe providing a pressure pump.

For yet another embodiment, provided is a method for using a constant force syringe pump enclosure assembly accommodating syringes of different sizes to dispense a solution from a syringe, including: providing a pump enclosure including: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base, the puller structured and arranged to receive a syringe having a plunger set to extrude a liquid from a chamber, the chamber having a length and a diameter; a sizer structured and arranged to determine the size of the chamber; an adjustable driver structured and arranged to provide a constant force between the pusher and the puller, the constant force adjustably selected by the size of the chamber; whereby slidably moving the puller distally when the syringe is seated in the base causes the pusher to contact and exert a substantially constant force specific for the size of the chamber on the head of the plunger, the constant force sufficient to move the plunger of the syringe; and an expandable cover connected to the proximal end of the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver; wherein the expandable base and expandable cover enclose the chamber body, the adjustable driver, puller and pusher are structured and arranged to provide force specific to the length of the chamber to the plunger of the syringe providing a pressure pump; opening the expandable cover of the pump enclosure; seating a selected syringe within the expandable base, the sizer determining a size of the selected syringe, the determined size selectively engaging adjustable driver; and closing the expandable cover to engage the pusher against the plunger of the selected syringe.

Further still, in yet another embodiment provided is a method for using a constant force syringe pump enclosure assembly accommodating syringes of different sizes to dispense a solution from a syringe, including: providing a pump enclosure comprising: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base, the puller structured and arranged to receive a syringe having a plunger set to extrude a liquid from a chamber, the chamber having a length and a diameter; a sizer structured and arranged to determine the size of the chamber; a plurality of different springs, each spring having a first end and a second end, the first end of each spring connected to the pusher, the second end of each spring selectively attached to the puller, the selection of at least one selectively attached spring from the plurality of springs based on the length of the chamber; whereby slidably moving the puller distally when the syringe is seated in the base causes the pusher to contact and exert a substantially constant force specific for the size of the chamber on the head of the plunger, the constant force sufficient to move the plunger of the syringe; and an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one selectively attached spring; wherein the expandable base and expandable cover enclose the chamber body, the at least one selectively attached spring, puller and pusher are structured and arranged to provide force specific to the length of the chamber to the plunger of the syringe providing a pressure pump; opening the expandable cover of the pump enclosure; seating a selected syringe within the expandable base, the sizer determining a size of the selected syringe, the determined size selectively engaging at least one spring from the plurality of springs; and closing the expandable cover to engage the pusher against the plunger of the selected syringe.

### BRIEF DESCRIPTION OF THE DRAWINGS

FIG. 1A is a upper perspective view of a Constant Force Pump For Different Syringe Sizes in an open condition in accordance with at least one embodiment of the present invention;

FIG. 1B is a upper perspective view of a Constant Force Pump For Different Syringe Sizes in an open condition further showing the adjustable driver including a plurality of springs in accordance with at least one embodiment of the present invention;

FIGS. 2A-2EE present simplified conceptualizations of an at least one embodiment of a sizer of the Constant Force Pump For Different Syringe Sizes for determining the size of a syringe chamber in accordance with at least one embodiment of the present invention.

FIGS. 3A-3DD present simplified conceptualizations of yet another embodiment of a sizer of the Constant Force Pump For Different Syringe Sizes for determining the size of a syringe chamber in accordance with at least one embodiment of the present invention.

FIGS. 4A-4C provide enlarged upper perspective section views of the control rods and engagers of a sizer in operation to engage one or more springs between a pusher and puller of a Constant Force Pump For Different Syringe Sizes in accordance with at least one embodiment of the present invention;

FIG. 5 provides an enlarged elevated section view of the puller and pusher and engagement elements of a Constant Force Pump For Different Syringe Sizes in accordance with at least one embodiment of the present invention;

FIGS. 6A-6C provide side views of the control rods and engagers of a sizer corresponding to FIG. 6 in operation to engage one or more springs between a pusher and puller of a Constant Force Pump For Different Syringe Sizes in accordance with at least one embodiment of the present invention;

FIG. 7 provides an enlarged elevation view of an alternative puller and pusher and engagement elements of a Constant Force Pump For Different Syringe Sizes in accordance with at least one embodiment of the present invention;

FIGS. 8A-8C provide side views of the control rods and engagers of a sizer corresponding to FIG. 7 in operation to engage one or more springs between a pusher and puller of a Constant Force Pump For Different Syringe Sizes in accordance with at least one embodiment of the present invention;

FIG. 9 provides an upper perspective view of a Constant Force Pump For Different Syringe Sizes with the cover in a partially closed condition in accordance with at least one embodiment of the present invention;

FIG. 10 provides a top view of a Constant Force Pump For Different Syringe Sizes in accordance with at least one embodiment of the present invention;

FIG. 11 provides a flow diagram of a method of using a Constant Force Pump For Different Syringe Sizes for infusion therapy.

### DETAILED DESCRIPTION

Before proceeding with the detailed description, it is to be appreciated that the present teaching is by way of example only, not by limitation. The concepts herein are not limited to use or application with a specific system or method for a compact mechanical syringe pump for dispensing liquids. Thus, although the instrumentalities described herein are for the convenience of explanation shown and described with respect to exemplary embodiments, it will be understood and appreciated that the principles herein may be applied equally in other types of systems and methods involving a compact mechanical syringe pump. Moreover, the features described or illustrated in connection with one embodiment may be combined with other embodiments, and such combinations, modification and variations are intended to be within the scope of the present invention.

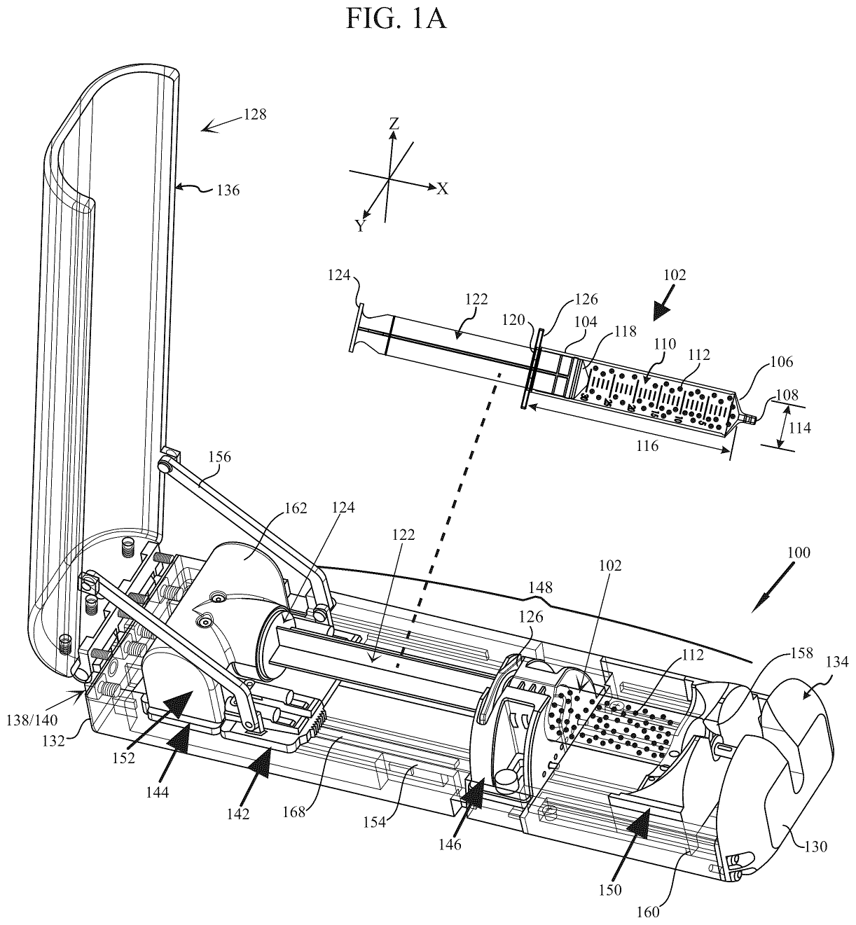

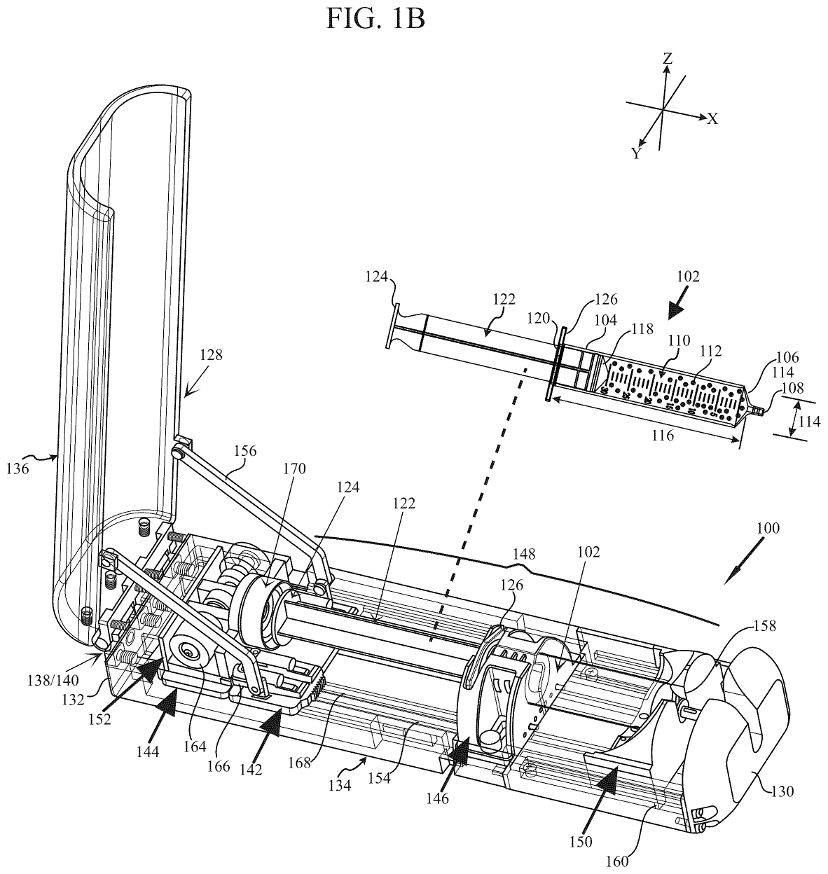

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIGS. 1A-1B there is shown a mechanical constant force pump as a Constant Force Pump For Different Syringe Sizes, hereinafter CFPDSS 100, for dispensing liquids, e.g., therapeutic liquids, from a variety of different sized syringes in accordance with at least one embodiment of the present invention. More specifically, CFPDSS 100 advantageously determines the size of the syringe so as to apply a proper and predetermined force according to the determined size.

To facilitate the description of systems and methods for embodiments of CFPDSS 100, the orientation of CFPDSS 100 as presented in the figures is referenced to the coordinate system with three axes orthogonal to one another as shown in FIG. 1A. The axes intersect mutually at the origin of the coordinate system, which is chosen to be the center of CFPDSS 100, however the axes shown in all figures are offset from their actual locations for clarity and ease of illustration.

As stated, CFPDSS 100 is structured and arranged to advantageously operate with a variety of different sized syringes, of which syringe 102 is merely exemplary. For ease of discussion and illustration, syringe 102 has been illustrated both disposed within the CFPDSS 100, and removed for ease of identification of various elements.

It will be understood and appreciated that, in general, a typical preloaded syringe 102 for use in an infusion treatment may be generally described as having a syringe barrel 104 which at a first end 106 provides an outlet 108 suitable for attachment to, or already fitted with, tubing suitable for administering the liquid in the syringe barrel 104. Normally, this first end of 104 the syringe 102 is tapered so as to reduce the overall size of the syringe 102 from the diameter of the barrel 104 to one suitable for fluid connection with tubing.

The barrel 104 of the syringe 102 also defines the chamber 110 of the syringe 102 which contains the liquid medication (shown as dots 112) to be dispensed. The diameter 114 of the chamber 110 is generally consistent along its length 116, and in most cases the length 116 of the chamber 110 and the diameter 114 of the chamber 110 are substantially the same as the diameter and length of the syringe barrel 104, save for the addition of the thickness of the material from which the syringe barrel 104 is formed. Moreover, unless specifically stated otherwise, it will be understood and appreciated that for the advantageous CFPDSS 100 as set forth and described herein, the diameter 114 of the barrel 104 and diameter 114 of the chamber 110 may be used interchangeably, likewise the length 116 of the barrel 104 and length 116 of the chamber 110 may be used interchangeably

A plunger seal 118 is disposed in the open end 120 of the syringe barrel 104, opposite from the first end 106. A plunger 122 extends rearward from the plunger seal 118 and is typically about the same length as the syringe barrel 104. As pressure is applied to the head 124 of the plunger 122, the plunger seal 118 is advanced towards the first end 106 of the syringe 102, and the liquid (shown as dots 112) within the syringe 102 is expelled through the outlet 108.

In general, a syringe 102 provides finger tabs 126 disposed proximate to the open end 120 of the syringe 102 such that a user may grasp the tabs with his or fingers and then exert pressure upon the head of the syringe 102 with his or her thumb, palm, or other hand. In other words, the finger tabs 126 provide a brace for the syringe barrel 104 as the plunger seal 118 is driven into and through the syringe barrel 104 by the plunger 122.

As useful as the finger tabs 126 may be, it will be understood and appreciated that the first end 106 of the syringe 102 may also be used as a base during the extrusion process. In some cases, the first end 106 may be fitted with a luer, such as a disc luer, in which case the disc portion of the luer may act as the base during the extrusion process. For at least one embodiment, the luer 200 is a flared luer as set forth in U.S. Pat. No. 10,500,389 entitled, SYSTEM AND METHOD FOR FLARED LUER CONNECTOR FOR MEDICAL TUBING, incorporated herein by reference. For yet another embodiment, the lure 200 is a tapered luer as set forth in U.S. Provisional Application 63/616,368 entitled, SYSTEM AND METHOD FOR A TAPERED LURE CONNECTOR FOR MEDICAL TUBING, incorporated herein by reference.

Moreover, to extrude the liquid from the syringe 102, the syringe barrel 104 must be braced by some element and the plunger 122 must be braced. A force is then applied to move the plunger 122 towards the first end 106. Although in hand held operation, the user may actually move the two components, it will also be understood and appreciated that the first end 106 can be pushed backwards towards the plunger 122, or the plunger 122 can be pushed forwards toward the first end 106.

In simple terms, it is an advantageous aspect of the CFPDSS 100 to receive a syringe 102 as has just been described, brace the first end 106 of the syringe 102 and brace the head 124 of the plunger 122, and then apply a constant force as between the first end 106 and the head 124 of the plunger 118 so as to drive the plunger seal 118 forward and extrude the liquid from the syringe 102. It will also be understood and appreciated, that for at least one alternative embodiment, the syringe 102 is braced by the finger tabs 126 rather than the first end 106. For such an embodiment, as a constant force is applied to the head 124 of the plunger 122, the bracing by the finger grips 126 ensures that the plunger seal 118 is driven forward towards the first 106 to extrude the liquid from the syringe 102.

For at least one embodiment, the CFPDSS 100 builds from the Applicant's prior developments with compact mechanical pumps as set forth in U.S. Pat. No. 10,376,636, which is incorporated herein by reference. However, whereas the ‘636 patent specifically teaches that if a person should try to dispose a 30 ml syringe in a pump suited for a 20 ml Syringe, ejection ramps will eject the Syringe from pump housing when the user attempts to close the housing, the present invention advantageously accommodates syringes 102 of different sizes.

Returning to FIG. 1A, for at least one embodiment, the CFPDSS 100 has a housing 128 having a distal end 130 and a proximal end 132. For at least one embodiment, the housing 128 is provided by a base 134 and a cover 136 that are hinged at the proximal end 132 to permit opening and closing. For at least one embodiment, the base 134 and a cover 136 are fixed in size. For yet another embodiment, the base 134 and a cover 136 are both expandable.

For at least one embodiment of an expandable CFPDSS 100, the base 134 is provided by a first base section and a second base section, wherein the first base section is in sliding engagement with the second base section such that the first base section and the second base section are slidably movable relative to each other between a compacted position and an expanded position. Likewise, the cover 136 is provided by a first cover section and a second cover section, wherein the first cover section is in sliding engagement with the second cover section such that the first cover section and the second cover section are slidably movable relative to each other between a compacted position and an expanded position. In general, the base 134 and cover 136 transition between compact and expanded states at the same time. Embodiments of such a compacting/expanding base 134 and cover 136 may be extrapolated from ‘636 patent noted above.

For at least one embodiment, the CFPDSS 100 also includes a dampener 138 to dampen the opening motion of the cover 136. In varying embodiments, the dampener 138 may be separate from the hinge 140 and pivotally coupled to base 134 and a cover 136, or integrated as an element of the hinge 140. Whether a separate element from the hinge 140 or an incorporated part of the hinge 140, for at least one embodiment the dampener 138 is selected from a viscus dampener, a fluid dampener, a rotary friction dampener or the like. Further still, in varying embodiments, the CFPDSS 100 may include a latch or lock assembly for keeping the cover 136 in a closed position upon the base 134.

Within the housing 128, and more specifically disposed upon the base 134 are a puller 142, a pusher 144, a collar 146, a sizer 148, and a selector 150. There is also an adjustable driver 152 structured and arranged to provide a constant force between the pusher 144 and the puller 142, this constant force adjustably selected by the size of the syringe 102, and more specifically the size of the chamber 110 of the syringe 102. It will be understood and appreciated that the size of the chamber 110 may be determined generally in two ways—by determining the length 116 of the chamber 110 or by determining the diameter 114 of the chamber 110.

It will be appreciated that for at least one embodiment, the pusher 144, the puller 142 and the selector 150 are slidingly engaged upon a track 154 or tracks provided by the base 134. It will also be appreciated that the puller 142 has movable linkage 156, linking the puller 142 with the cover 136, such that movement of the cover 136 between an open and closed position imparts sliding motion of the puller 142 along the track(s) of the base 134. More specifically, as the cover 136 is lowered for closing, the linkage 156 moves the puller 142 towards the distal end 130, and when the cover 136 is raised for opening, the linkage 156 moves the puller 142 towards the proximal end 132.

Although for the conceptual embodiment as shown, the cover 136 and base are pivotally connected at one end, an alternative embodiment may be provided where the cover 136 slides longitudinally over the base, the movable linkage 156 linking the puller 142 with the cover 136 being mechanically adapted for activation through the sliding of the cover 136 between an open and closed position as oppose to rotation.

As the CFPDSS 100 is advantageously structured and arranged to accommodate a plurality of different syringe sizes—e.g., a syringe having a volume of at least 5 milliliters to a syringe having 120 milliliters—it will be understood and appreciated that CFPDSS 100 advantageously permits a different constant force to be applied to each of the different sized syringes. It is this different constant force that is provided by the adjustable driver 152.

For at least one embodiment of CFPDSS 100, the adjustable driver 152 is provided, at least in part, by a plurality of different springs that may be selectively engaged between the pusher 144 and the puller 142. It will be understood and appreciated that other options for an adjustable driver 152 structured and arranged to provide a constant force between the pusher 144 and the puller 142 that is determined by the size of the syringe 102. One such alternative embodiment is provided by at least one spring engaged between the pusher 144 and the puller 142 and at least one braker associated with the at least one spring (not shown)—the braker providing an adjustable braking force to the at least one associated spring, the braking fore applied based on the size of the syringe 102 as determined by the size of the chamber.

Returning to FIG. 1A, and at least a first embodiment of a CFPDSS 100, for at least one exemplary embodiment as shown, the collar 146 is disposed in a fixed position upon the base 134, however it will be understood and appreciated that in at least one alternative embodiment the collar 146 may also be slidingly engaged upon a track or tracks provided by the base 134.

For the accommodation of different syringes 102 having different barrel diameters, in at least one embodiment the collar 146 may be provided by essentially two side elements that may be adjusted by the user outward or inward so as to firmly grip the syringe barrel 104. Various alternatives such as, but not limited to, compliant materials that will compress or expand as required by the diameter of the syringe barrel 104 may also be used, so as to permit the collar 146 to firmly/snuggly grip the syringe barrel and/or provide brace support for the finger tabs of the syringe 102.

For ease of discussion, a syringe 102 is shown as disposed within the CFPDSS 100. More specifically the finger tabs at the open end of the syringe barrel 104 are shown to be engaged with the collar 146, and the first end 106 of the syringe 102 is shown seated with the selector 150.

The selector 150 is selectively movable between multiple positions, which for at least one embodiment are the varying, and distinctly different barrel lengths of a 50 ml syringe 102, a 20 ml syringe 102, a 10 ml syringe 102, and a 5 ml syringe 102.

For at least one embodiment, a spring may bias the selector 150 to be disposed proximate to the collar 146. As a selected syringe 102 is disposed in the CFPDSS 100—the first end 106 of the syringe 102 is partially disposed in a seat 158 provided by the selector 150. As the syringe 102 is lowered down so as to engage the finger tabs 126 with the collar 146, the selector 150 is slid away from the collar 146 to a distinct position appropriate for the size of the syringe 102.

As noted above, for varying embodiments, where the syringe is coupled to a lure, such as the flared luer as set forth in U.S. Pat. No. 10,500,389 entitled, SYSTEM AND METHOD FOR FLARED LUER CONNECTOR FOR MEDICAL TUBING or a tapered luer as set forth in U.S. Provisional Application 63/616,368 entitled, SYSTEM AND METHOD FOR A TAPERED LURE CONNECTOR FOR MEDICAL TUBING, the selector 150, and more specifically the seat 158 of the selector may be structured and arranged as an accepting base for such a tapered or flared luer.

For this present example embodiment, the size of the syringe 102 is determined by length 116 of the syringe 102, and more specifically the length 116 of the syringe barrel 104/chamber 110. In such an embodiment, it will be understood and appreciated that the collar 146, which has received the finger tabs 126 and is bracing them is providing the brace for the syringe 102, much as the fingers of a human operator would. For yet another embodiment, the collar 146 and the selector 150 both provide brace for the syringe barrel 104.

It will be understood and appreciated that for at least one embodiment it may be the selector 150 which provides the brace for the syringe 102 in place of the collar 146. Moreover, for at least one embodiment, each distinct position has a binder 160 such that once the selector 150 is slid to a desired position it may be temporarily bind to the base 134 at that position. For at least one embodiment the binder 160 is provided by notches in the base 134 and ridges extending from the selector 150. When the selector 150 is grasped and tilted slightly up, the ridges are removed from the notches and the selector 150 may be moved to a new position. Once repositioned, the selector 150 is pressed down and the ridges are then depressed into corresponding notches such that the selector 150 will remain in the chosen position until it is tilted up once again to disengage the ridges from the notches.

For at least one embodiment, the selector 150 is a component of a sizer 148—which is structured and arranged to determine the size of the chamber of the syringe 102 disposed in the CFPDSS 100, and more specifically seated between the selector 150 and the collar 146. For at least one embodiment, the sizer 148 is a mechanical system provided at least in part by the selector 150 and at least one selector or control rod or bar that mechanically interconnects the selector 150 with the puller 142.

In FIG. 1A, the adjustable driver 152 is shown with a covering 162 in place. In FIG. 1B, the cover 162 has been removed, and for the exemplary embodiment shown, the adjustable driver 152 is appreciated to be a plurality of tape springs 164, of which tape springs 164A, 164B, 164C and 164D are exemplary and shown in FIG. 1B.

With respect to interconnecting the selector 150 with the puller, it will be appreciated in FIG. 1B that, for at least one embodiment, the selected position of the selector 150 mechanically triggers one or more control rod(s)/bar(s) 168 to engage and selectively attach the first end 166 of one or more tape springs 164 to the puller 142. Indeed, each of the one or more control rod(s)/bar(s) 168 may be further described as a lever-having a low section and a high section. As the selector 150 is slid longitudinally within the housing 128 along one or more tracks 154, the one or more control rod(s)/bar(s)/lever(s) 168 may be slid a sufficient distance such that the low section transitions to a high section, the high section activating the selection of an associated tape spring 164.

Indeed, it will be understood and appreciated that mechanical determination of the size of the syringe 102 is, for at least one embodiment determined by the movement of the selector 150 towards the distal end 130 as the syringe 102 is disposed into the CFPDSS 100. More specifically, the user disposes the first end 106 of the syringe 102 into the seat 158 of the selector 150 and pushes the selector 150 towards the distal end 130 so as to bring the finger tabs 126 into position to be disposed into the collar 146. The longitudinal movement of the selector 150 within the housing 128 along the base 134 is triggers one or more control rod(s)/bar(s)/lever(s) to selectively couple one or more tape springs 164 provided by the pusher 144 to the puller 142. As such, unless specifically described otherwise, it will be understood and appreciated that the terms “rod” /“bar” /“lever” with respect to the controlling linkage triggering the engagement of the first end 166 of a selected tape spring 164, may be used interchangeably.

When the cover 136 is closed, the linkage 156 coupled between the cover 136 and the puller 142 operates to drive the puller 142 down the track(s) 154 of the base towards the collar 146 and the distal end 130. As this sliding movement occurs, each tape spring 164 that has been selectively attached will be drawn out, thus providing a force of attraction between the pusher 144 and the puller 142, as the selectively attached tape springs 164 attempt to recoil themselves.

As the puller 142 will be held substantially in place by the linkage 156 when the cover 136 is closed, it is the pusher 144 which will then slide towards the puller 142. As may be appreciated in FIGS. 1A and 1B, the pusher 144 has a plunger receiver 170 which is structured and arranged to contact the head 124 of the plunger 122. For at least one embodiment, the plunger receiver 170 does not just contact, but engages with the head 124 of the plunger 122 such as by providing a seat or basin which may help to center the plunger 122 and avoid possible slide slippage when and as force is applied to the plunger 122. The tape spring(s) 164 imparted force drawing the pusher 144 towards the puller 142 is thereby applied to the plunger 122—driving it into the syringe barrel 104 and extruding the liquid from the outlet 108 at the first end 106.

Moreover, the sizer 148—provided by the exemplary embodiment of the selector 150, the control rod(s)/bar(s)/lever(s) 168 and the selected engagement of the first ends 166 of one or more tape springs 164 advantageously ensures that the force provided by the engaged tape springs 164 is a pre-determined forced appropriate for the size of the syringe 102 that has been disposed within CFPDSS 100.

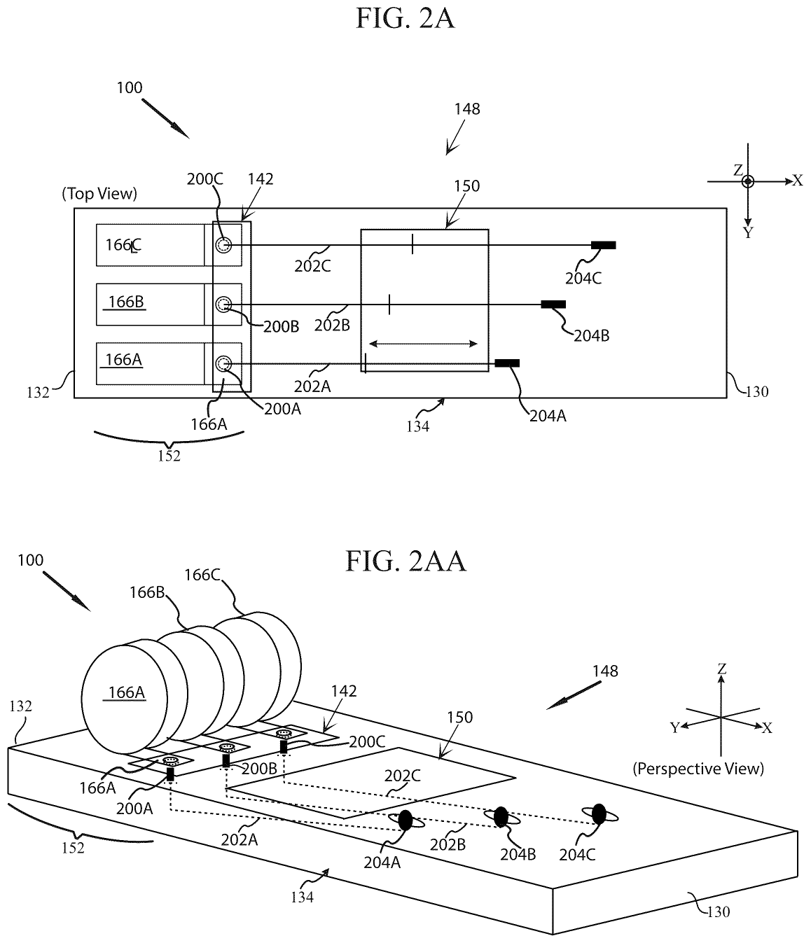

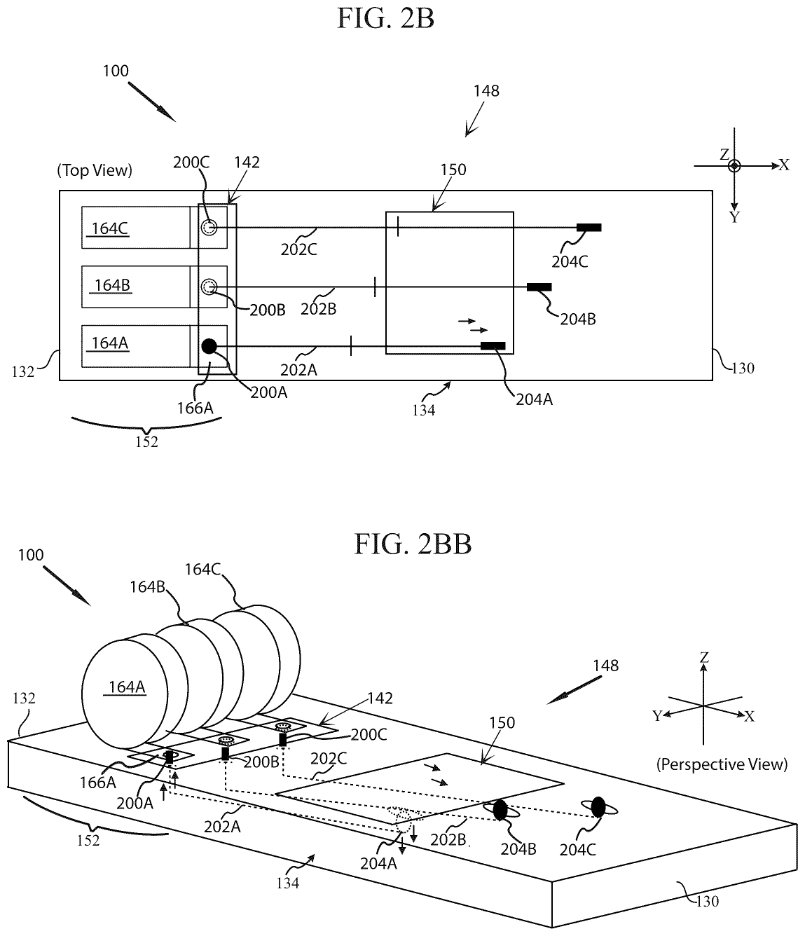

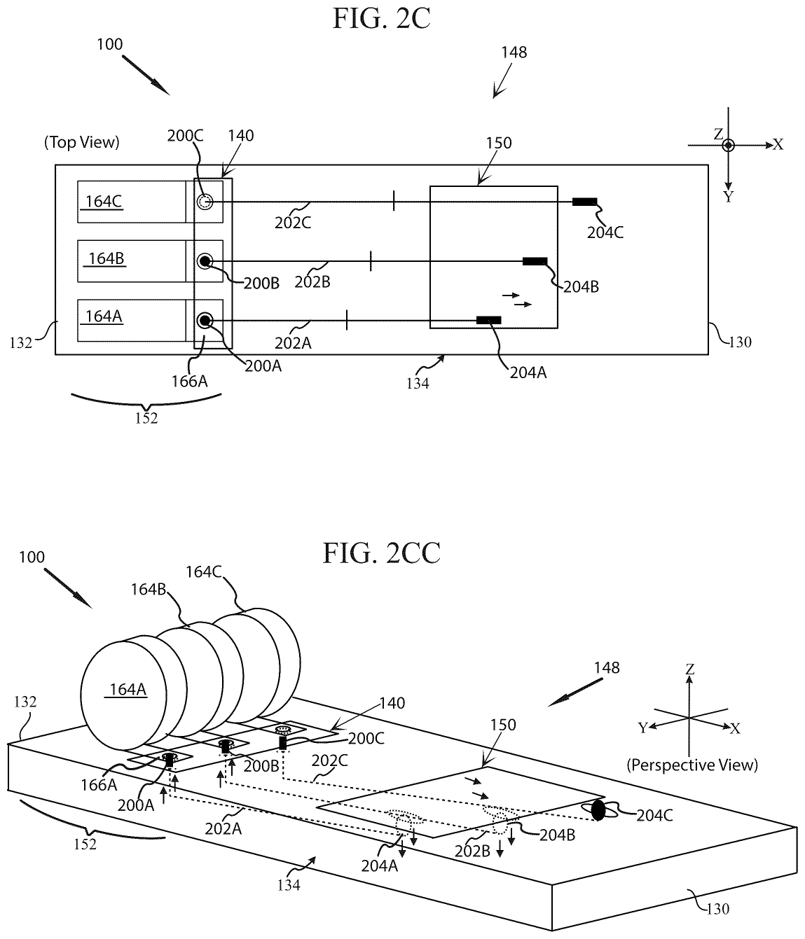

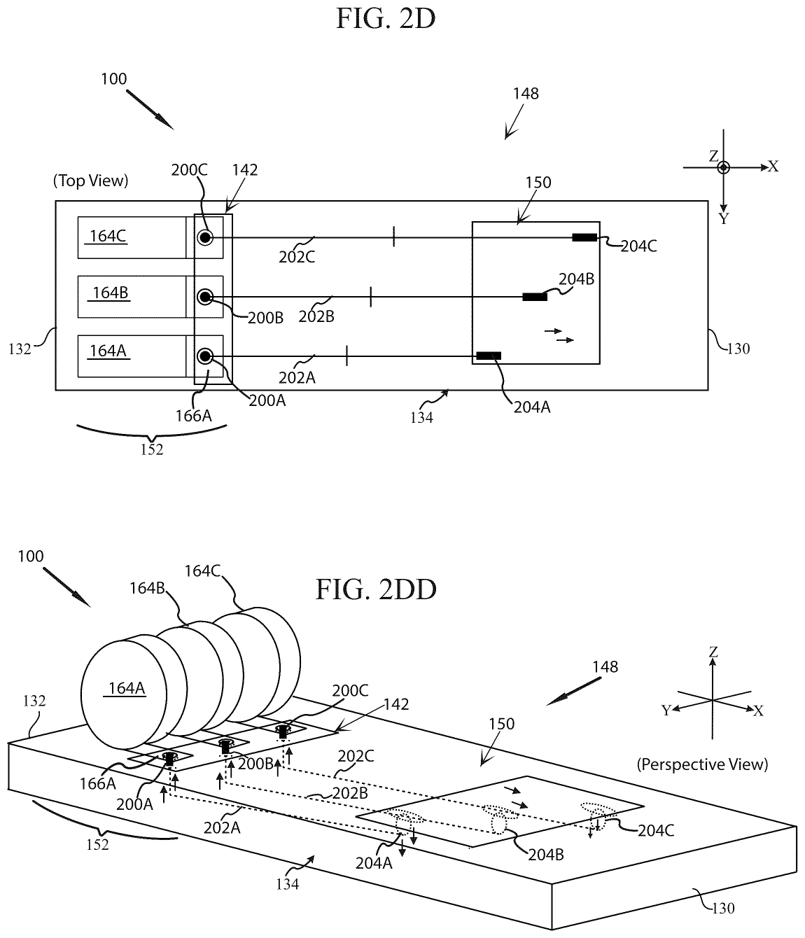

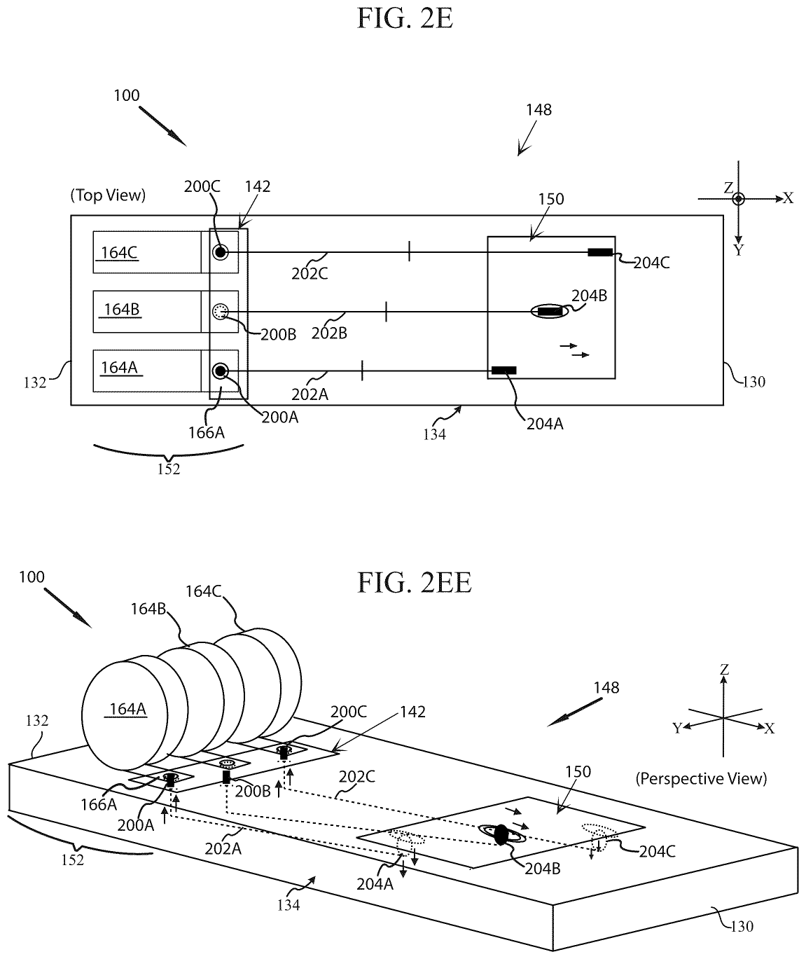

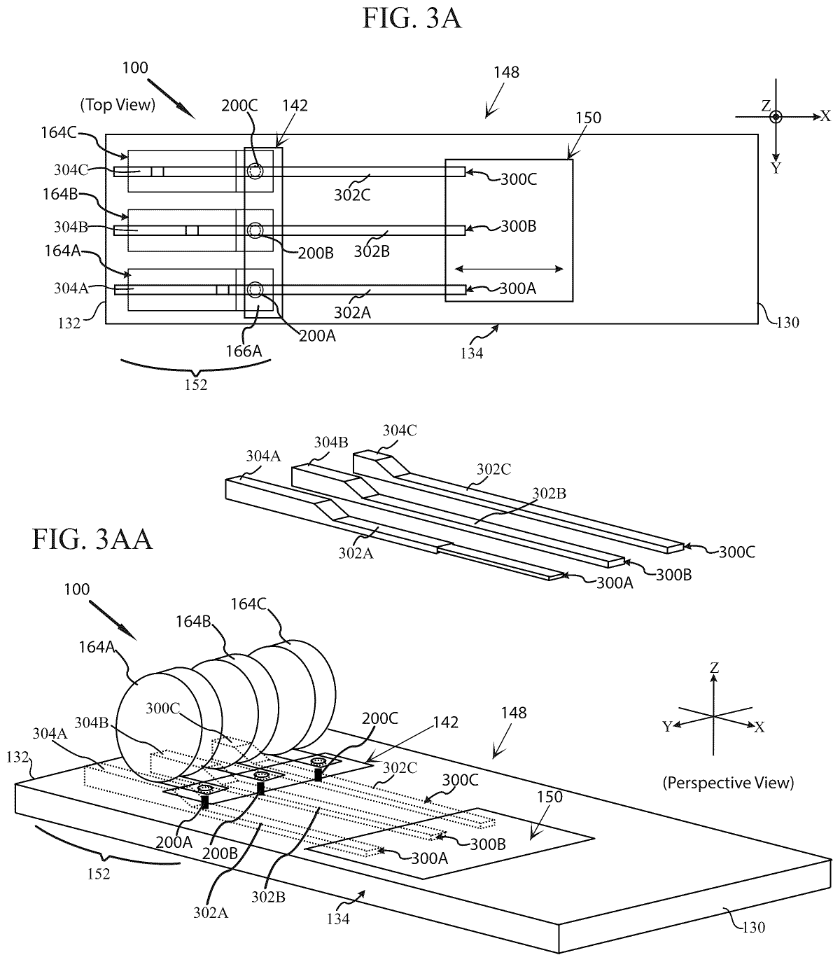

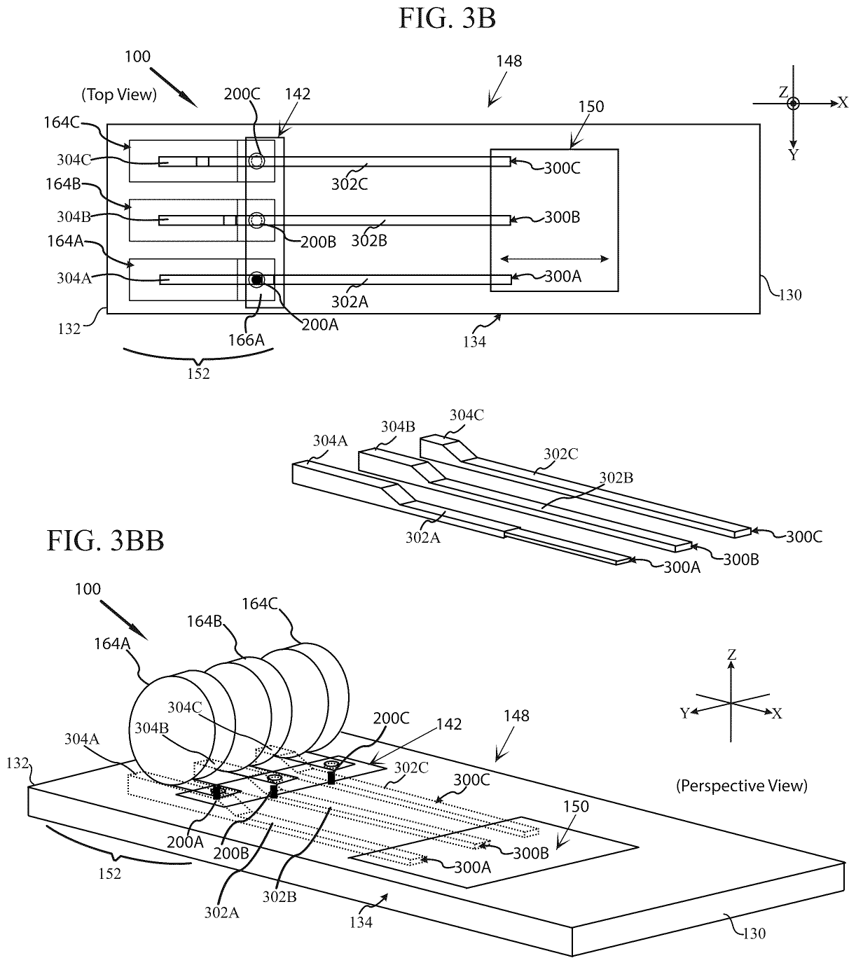

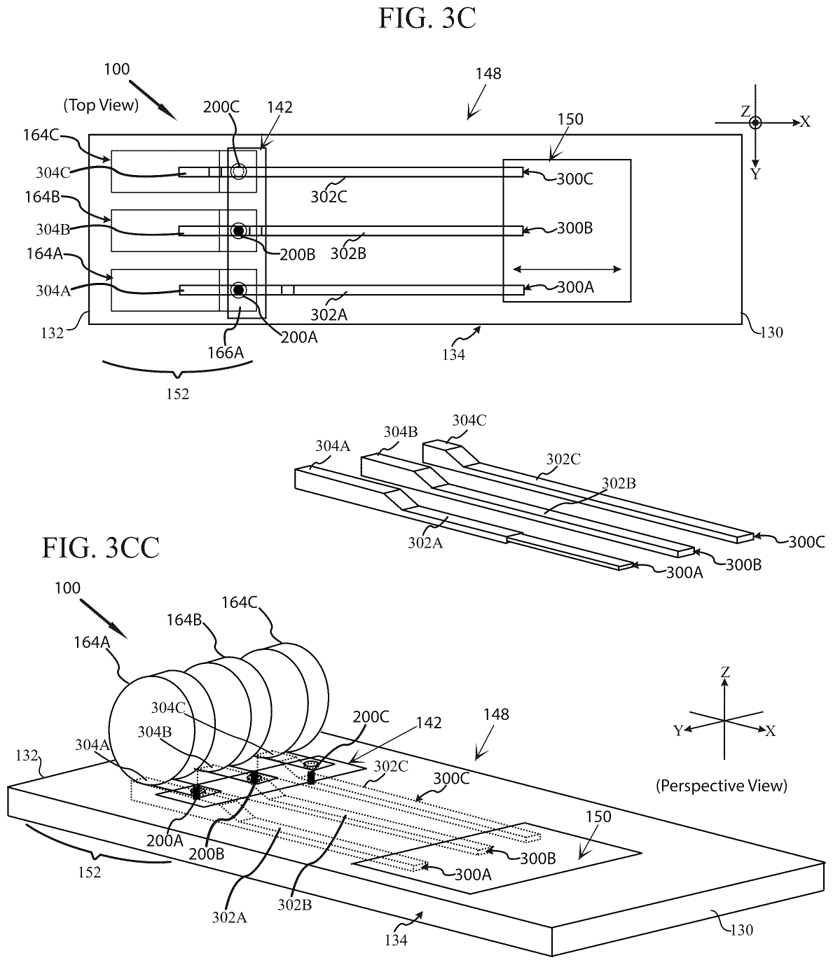

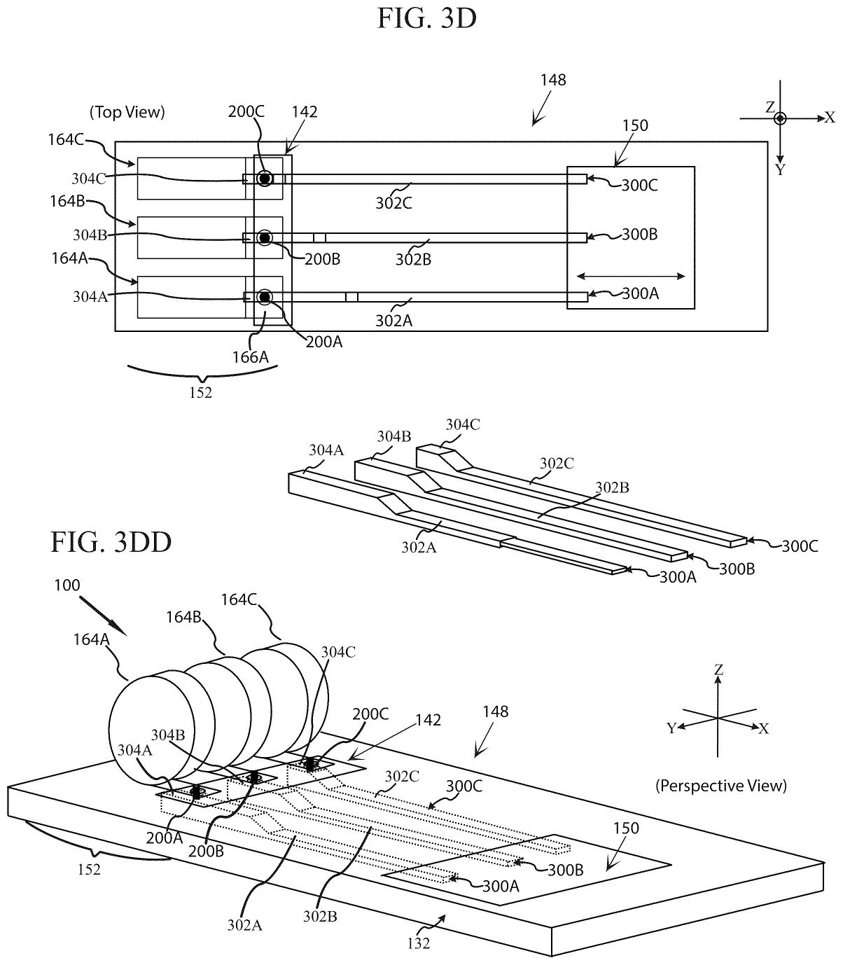

This mechanical selection of tape springs 164 based on position of the selector 150 may be more fully appreciated with respect to the conceptual illustrations presented in FIGS. 2A-2EE and FIGS. 3A-3DD. For ease of discussion and illustration the CFPDSS 100 system has been simplified for demonstration with three tape springs 164 and how they are selectively attached to the puller 142. It will be understood and appreciated from a review of at least FIG. 1B that the tape springs 164 are understood and appreciated to be disposed on the pusher 144, but for the sake of ease of illustration and discussion, the distinct element of the pusher 144 as well as the syringe 102, cover 136, and collar 146 have been omitted in FIGS. 2A-2EE and 3A-3DD to help emphasize and clarify the conception of selective attachment of the tape springs 164 to the puller 142.

In each FIGS. 2A-2EE, at the top of the drawing page is shown a top view (FIGS. 2A, 2B, 2C, 2D, 2E) of simplified CFPDSS 100 and at the bottom is shown a corresponding perspective view (FIGS. 2AA, 2BB, 2CC, 2DD, 2EE) of simplified CFPDSS 100. In FIG. 2A the system is at rest, and none of the tape springs 164 have been engaged. FIGS. 2B & 2BB, 2C & 2CC, 2D & 2DD, and 2E & 2EE each illustrate how different syringes having different lengths advantageously mechanically trigger different engagements of tape springs 164, thus advantageously assuring and permitting CFPDSS 100 to provide the correct constant force to plunger 122 of syringe 102 disposed within CFPDSS 100.

More specifically, turning to FIGS. 2A & 2AA, three tape springs 164A, 164B, 164C are shown each with a first end 166 disposed over a puller 142. Below the puller 142 and first end 166 of each tape spring 164 is an engager 200, and each engager 200 is disposed upon the end of an associated control rod 168, which for the illustrated example is a lever 202. A selector 150 is shown disposed over the base 134, and each of the three levers 202A, 202B, 202C is shown to have an activator 204A, 204B, 204C.

In FIGS. 2B & 2BB, conceptualizing the insertion of a syringe having a first length, the selector 150 has been slid forward and has now depressed the activator 204A of the first lever 202A. With the activator 204A depressed, the other end of the lever 202A is elevated such that the engager 200A is now disposed to engage the first end 166A of tape spring 164A to the puller 142. It will be understood and appreciated that for this conceptual drawing, the engager 200A is shown as a distinct element, such that once engaged, it can move with the puller 142 and thereby maintain attachment between the puller 142 and tape spring 164A even when the puller 142 is slid away from the springs—as occurs when the cover is closed such that the linkage 156 (not shown in FIGS. 2B & 2BB) causes the puller 142 to slide away from the pusher 144.

In FIGS. 2C & 2CC, conceptualizing the insertion of a syringe having a second length, the selector 150 has been slid to a second position and is now so disposed that the first and second activators 204A & 204B are both triggered, and correspondingly the engagers 200A & 200B for both tape spring 164A and tape spring 164B have been elevated for temporary attachment of their respective first ends 166A & 166B to the puller 142.

In FIGS. 2D & 2DD, conceptualizing the insertion of a syringe having a third length, the selector 150 has been slid to a third position, and now all three activators 204A & 204B & 204C have been triggered such that all three tape springs 164A, 164B and 164C are temporarily attached by their respective engager 200A, 200B, 200C to the puller 142.

In FIGS. 2E & 2EE, conceptualizing the insertion of a syringe having a fourth length, a variation of the selector 150 has been shown wherein the first and third activators 204A & 204C are triggered, but the second activator 204B is not. As such, tape springs 164A and 164C are temporarily attached to the puller 142, but tape spring 164B is not.

Moreover, it will be understood that selectively engaging combinations of tape springs 164A, 164B, 164C, different spring force combinations may easily, reliably, and repeatably be achieved between the puller 142 and the pusher 144 to provide the proper pre-determined constant force to the syringe disposed within the CFPDSS 100, this proper constant force advantageously and automatically being adjusted by the CFPDSS 100 in accordance with the size of the syringe 102—e.g., the size of the chamber 110 of the syringe 102. In certain embodiments, the resulting constant force from the tape springs is about 1 to 25 pounds of force. In certain embodiments, the CFPDSS 100 adaptively provides pressure from about 5 psi to about 30 psi. In other words, CFPDSS 100 advantageously and adaptively changing combinations of tape springs 164 ensures that the selected pre-determined constant force is appropriate to the size of the chamber, thus ensuring an outflow rate from the syringe chamber 110 that does not exceed a pre-determined rate.

It will also be understood that in varying embodiments, each of the tape springs 164 may be substantially identical, at least two tape springs 164 may be substantially identical, and/or each tape spring 164 is distinctly different. More specifically, the use of at least four tape springs 164 may be selectively chosen so as to provide the proper constant force for a plurality of different, yet common, pre-filled syringes as may be desired and used in infusion therapy regimes.

As an alternative to the embodiment of levers depicted in FIGS. 2A-2EE, FIGS. 3A-3DD provide a simplified conceptual overview of an alternative embodiment of the tape spring 166 selection process as achieved by an embodiment of CFPDSS 100 incorporating tapered control rods 300. In each FIGS. 3A-3DD, at the top of the drawing page is shown a top view of CFPDSS 100 (FIGS. 3A, 3B, 3C, 3D) and at the bottom is shown a corresponding perspective view of CFPDSS 100 (FIGS. 3AA, 3BB, 3CC, 3DD). In the middle of this top and perspective view, a reference view of the control rods 168, which for the present illustrations are identified as control rods 300A, 300B, and 300C.

In each reference view, each control rod 300, and more specifically control rods 300A, 300B, and 300C is appreciated to have a low section 302A, 302B, and 302C and a high section 304A, 304B, and 304C. As may be appreciated from the reference illustrations, each control rod 300 has a different profile as each has a high section 304 of a different length.

In addition, control rod 300A has been illustrated with a telescoping slide arrangement such that a portion of the low section 302A may be slid into the portion of control rod 300A providing the high section 304A. Of course, it will be understood and appreciated that this arrangement may be reversed in an alternative embodiment wherein the portion of the control rod 300A providing the high section 304A slides into the portion of the control rod 300A providing the low section.

For either arrangement, it will be understood and appreciated that these alternative embodiments of control rods 300 would be incorporated in alternative embodiments noted above where the base 134 and cover 136 are also configured to have a sliding arrangement as between a compact for storage and extended for use state of configuration.

As in FIGS. 2A & 2AA, in FIGS. 3A & 3AA, the simplified CFPDSS 100 is at rest-each of the control rods 300A, 300B, 300C is disposed in an initial position under the tape springs 164A, 164B, 164C such that all of the high sections 304A, 304B, and 304C are away from the engagers 200A, 200B, 200C, which are at present disposed above the low section 302A, 302B, and 302C of each control rod 300A, 300B, 300C.

In FIGS. 3B & 3BB, conceptualizing the insertion of a first syringe having a first length, the selector 150 has been advanced longitudinally away from the tape springs 164 to a first position for a first syringe, and the first selector rod 300A has now moved as well such that the high section 302A is now disposed below the engager 200A which has been lifted up so as to engage tape spring 164A to the puller 142.

In FIGS. 3C & 3CC, conceptualizing the insertion of a syringe having a second length, the selector 150 has advanced longitudinally away from the tape springs 164 to a second position for a second syringe which is of a size different from the first syringe. At this second position, both the first and second control rods 300A & 300B have been moved a sufficient distance such that both high sections 304A & 304B are now disposed beneath engagers 200A & 200B such that both tape springs 164A & 164B are now selectively engaged to the puller 142.

In FIGS. 3D & 3DD, conceptualizing the insertion of a syringe having a third length, the selector 150 has advanced longitudinally away from the tape springs 164 to a third position for a third syringe which is of a size different from both the first syringe and the second syringe. At this third position, all three control rods 300A, 300B, 300C have been moved a sufficient distance such that all high sections 304A, 304B, 304C are now disposed beneath engagers 200A, 200B, and 200C such that all three springs are now selectively engaged to the puller 142. It will be understood and appreciated that yet at least a fourth option for at least a fourth length of a syringe may also be provided, corresponding at least in part to FIGS. 2E & 2EE where an aperture may be provided such that two or more control rods 300 are engaged while at least one control rod 300 is not.

With respect to the above description of FIGS. 2A-2E and FIGS. 3A-3D, and the description of the sizer 148 as a mechanical system provided at least in part by the selector 150 and at least one control rod that mechanically interconnects the selector 150 with the puller 142, it will be understood and appreciated that an alternative selector 150 embodiment may also be employed. More specifically, the selector 150 may be incorporated as an element of the collar 146.

As noted above, to accommodate syringes of different diameters 114, the collar 146 may be provided by essentially two side elements that may be adjusted by the user outward or inward so as to firmly grip the syringe barrel 104. It will be understood and appreciated that activation of the control rods 168/202/300 as described above may be achieved by linkage between the side elements of the collar and the control rods 168/202/300. Such linkage may be in the form of gears, arms, levers or other elements that are structured and arranged to translate lateral movement of the side elements of the collar 146 into longitudinal movement of the control rods 168/202/300.

Moreover, for at least one embodiment, the size of the syringe 102, and more specifically the chamber 110/barrel 104 is determined by the length 116 of the chamber 110 as determined by the longitudinal moving selector 150 as conceptualized in FIGs. FIGS. 2A-2E and FIGS. 3A-3D. For at least one alternative embodiment, the size of the syringe 102, and more specifically the chamber 110/barrel 104 is determined by the diameter 114 of the chamber 110 as determined by a selector 150 provided by laterally moving side elements of the collar 146.

In addition, although the above discussion had presented mechanical embodiments for the Sizer, it will also be understood and appreciated that sizer 148 may be an electrical system.

More specifically, for at least one alternative embodiment of CFPDSS 100, the sizer 148 incorporates a Hall effect sensor. More specifically, magnets are disposed into the base 134 and the selector 150 or collar 146. As the selector 150 and/or collar 146 are slid on one direction or another, magnets attached thereto are sensed by a Hall effect sensor array, as may be provided by a printed circuit board (PCB). This sensed signal is interpreted by one or more computer chips utilizing a position algorithm to determine the length of the syringe barrel, the computer chips then triggering one or more engagers 200 (such as for example solenoids) to selectively attach one or more tape springs 166 to the puller 142.

For yet another embodiment of CFPDSS 100, the sizer 148 is provided at least in part by a resistive strip. Here again, movement of the selector 150 or collar 146 is detected by a wiper moving along a resistive strip. This sensed change in resistance is interpreted by one or more computer chips utilizing a position algorithm to determine the length of the Syringe barrel, the computer chips then triggering one or more engagers 200 (such as for example solenoids) to selectively attach one or more tape springs 166 to the puller 142.

Still for yet another embodiment of CFPDSS 100, the sizer 148 is provided is provided at least in part by a laser system measuring time of flight (TOF). In general, a laser beam is activated to strike a target mounted on an element that is understood and appreciated to move so as to properly accommodate a syringe—e.g. the selector 150 or collar 146. Laser light reflected back by the target is captured by a sensor. This time elapsed between emission of the laser and sensing of the bounced return is interpreted by one or more computer chips utilizing a position algorithm to determine the length of the syringe barrel, the computer chips then triggering one or more engagers 200 (such as for example solenoids) to selectively attach one or more tape springs 166 to the puller 142.

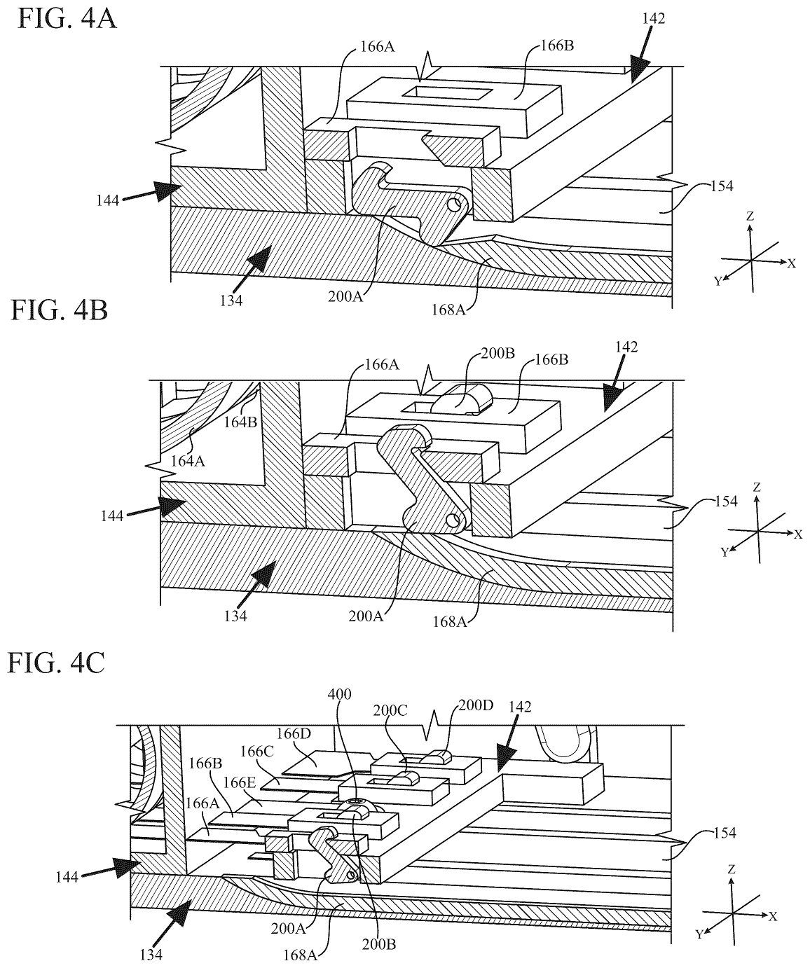

FIGS. 4A-4C present enlarged partial illustrations of the control rods 168 and engagers 200, and their progression of operation. More specifically, in FIG. 4A the CFPDSS 100 is at rest and no syringe has yet been disposed engaged to the collar and selector (not shown). In FIG. 4B, a syringe has now been disposed within CFPDSS 100, and the selector has been moved from a first position at rest to a second position now indicative of the length of the syringe barrel.

As such, this movement of the selector 150 has triggered the movement of at least one control rod 168, and as shown clearly, exemplary control rod 168A has moved to lift the engager 200A so as to engage the first end 166A of tape spring 164A to the puller 142. As is shown in FIG. 4B, multiple control rods have been engaged resulting in at least engagers 200A and 200B engaging first ends 166A & 166B of their associated tape springs 164A & 164B.

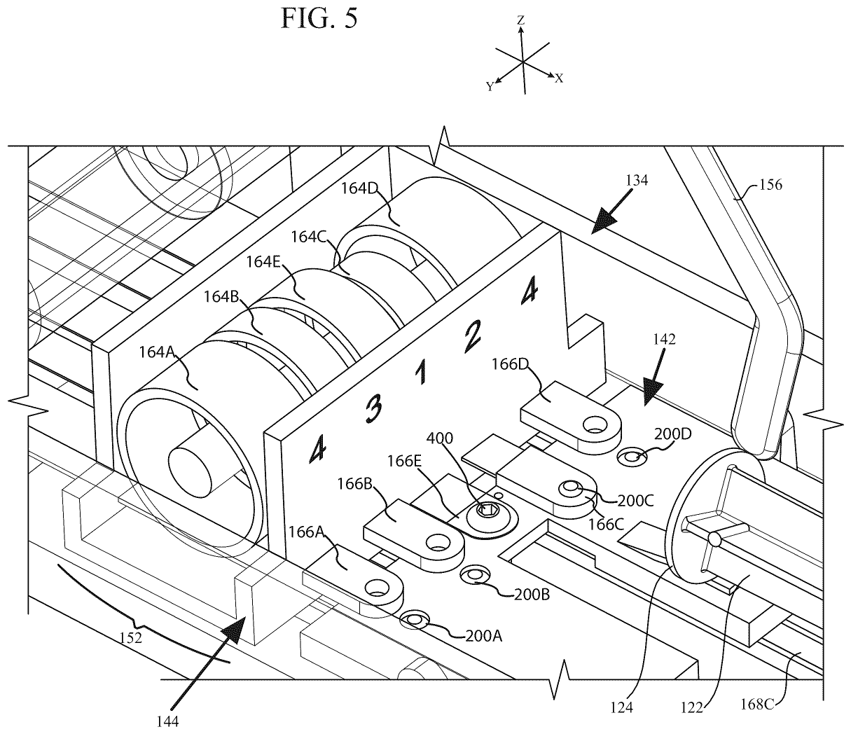

In FIG. 4C, the puller 142 has been slid away from the pusher 144 and it may be appreciated that all four (4) of the selectable tape springs 164A, 164B, 164C and 164D have been engaged by their first ends 166A, 166B, 166C and 166D to the puller 142. It may also be appreciated that for at least one embodiment, one tape spring, such as 164E (not shown in FIG. 4C—see FIG. 5A) is permanently affixed between the puller 142 and the pusher 144. For the embodiment as shown, this is the middle tape spring 164E, the first end affixed to the puller 142 by a fixed attacher 400, such as a screw.

FIG. 5 is an elevated partial perspective showing a similar subsection of CFPDSS 100 as show in in FIG. 4C, FIG. 5 illustrated from a higher view angle. In FIG. 5 the plunger receiver and other elements have been removed so as to provide an enhanced view of the engagement between the puller 142 and the first ends 166 of at least some tape springs 164. The condition of tape spring 164E as permanently attached between the puller 142 and the pusher 144 is more clearly illustrated. In addition, it may also be appreciated that for the exemplary embodiment shown, tape spring 164C has been selectively engaged to the puller 142, but tape spring 164A, 164B and 164D have not. More specifically, engager 200C is shown engaging with first end 164C, but engagers 200A, 200B, 200D are shown in their undeployed, and therefore non-engaging state.

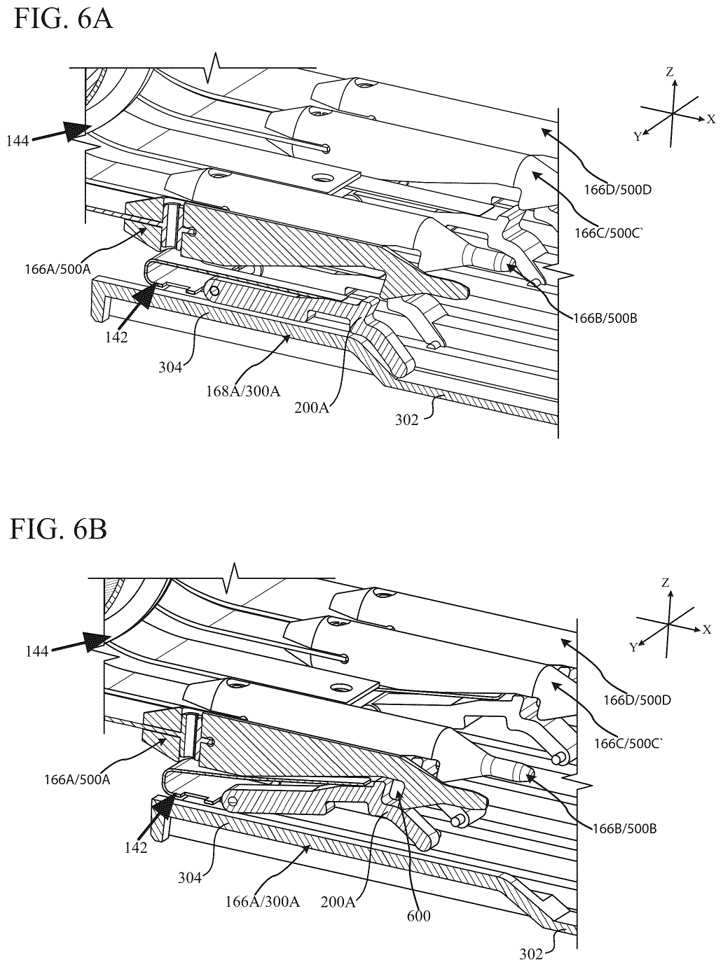

FIGS. 6A-6C present enlarged partial illustrations of yet another embodiment of CFPDSS 100 wherein the control rods 168 are tapered control rods 300 having low sections 302 and high sections 304 as discussed above with respect to FIGS. 3A-3DD, and their progression of operation.

For this embodiment, each control rod 168/300 is moved longitudinally within the housing as the selector 150 is moved longitudinally. The selection of a specific tape spring is achieved by its associated control rod 168 moving a sufficient lateral distance to transition from the low section 302 to the high section 304, such that the engager 200 is pressed up so as to engage the first end 166 of its associated tape spring 164.

It should also be noted that each control rod 168 as shown in FIGS. 6A-6C may be a telescoping control rod or bar as discussed above. When the CFPDSS 100 is compacted, the telescoping nature of each control rod 168 permits a first portion to move within the second portion (or vice versa) but when the CFPDSS 100 is expanded so as to receive a syringe, the first portion locks with the second portion to provide the full-length control rod 168. In at least one embodiment springs (not shown) may be disposed between the second portion and the hinge end of the CFPDSS 100 to retard lateral motion of the second portion of each control rod unless and until the first portion has fully retracted.

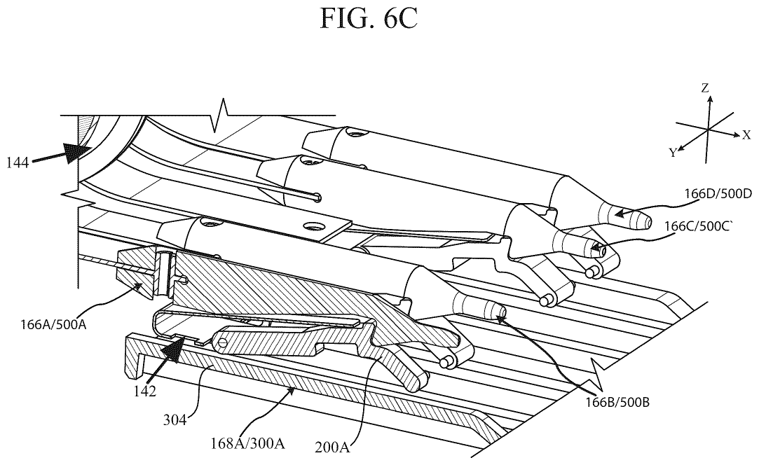

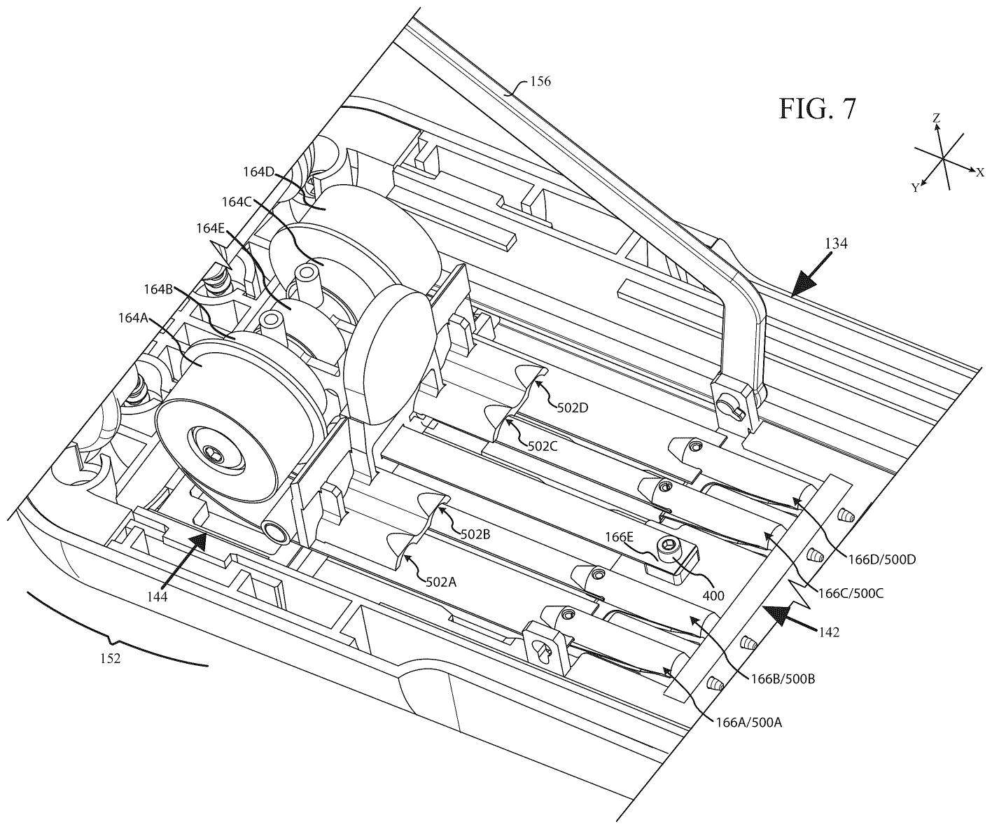

In addition, the illustrated embodiment utilizes the tapered pins 500 as the first ends 166 of the tape springs 164 (see FIG. 7 for tape springs 164). More specifically, in FIG. 6A the CFPDSS 100 is at rest and no syringe 102 has yet been disposed engaged to the collar 146 and selector 150 (not shown).

In FIG. 6B, a syringe 102 (not shown) has now been disposed within CFPDSS 100, and the selector 150 (not shown) has been moved from a first position at rest to a second position now indicating the length of the syringe barrel. As such, this movement of the selector 150 has triggered the movement of at least one control rod 168, and as shown clearly, exemplary control rod 168A/300A has moved such that the high section 304 is now below the engager 200A so as to lift the engager 200A to engage the first end 166A/500A of tape spring 164A to the puller 142. As this is the initial movement for engagement, there is shown a gap 600 between the engager 200A and the first end 166A/500A of tape spring 164A (see FIG. 7).

In FIG. 6C, the puller 142 has been slid away from the pusher 144 and it may be appreciated that selectable tape springs 164A has been engaged by first end 166A/500A to the puller 142 as engager 200A is now in direct contact with the first end 166A/500A of tape spring 164A.

FIG. 7 shows a partial perspective view of an embodiment of CFPDSS 100 utilizing tapered pins 500 as the first ends 166 of the selectable tape springs 164, and sockets 502 for advantageously improved alignment and engagement of the first ends 500/166. As with partial perspective FIG. 5 some elements (e.g., plunger, plunger receiver . . . ) have been removed to permit an enhanced view and appreciation of at least one alternative embodiment for engaging tape springs 164 between the puller 142 and pusher 144. More specifically, tapered pins 500 self-align to their corresponding receiving sockets 502. As they are self-aligned, the engagement of an engager 200 (not shown in FIG. 7) for the selection of a tape spring 164 is likely improved.

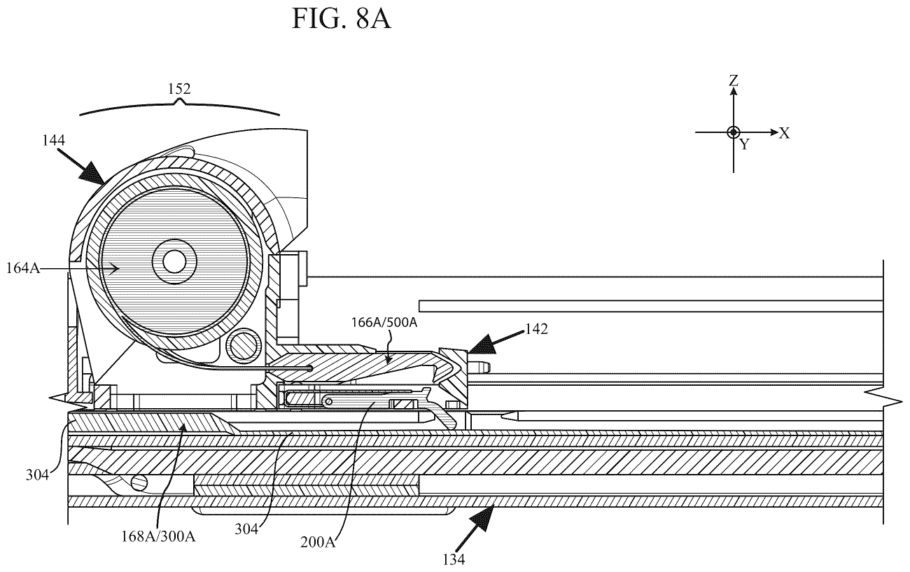

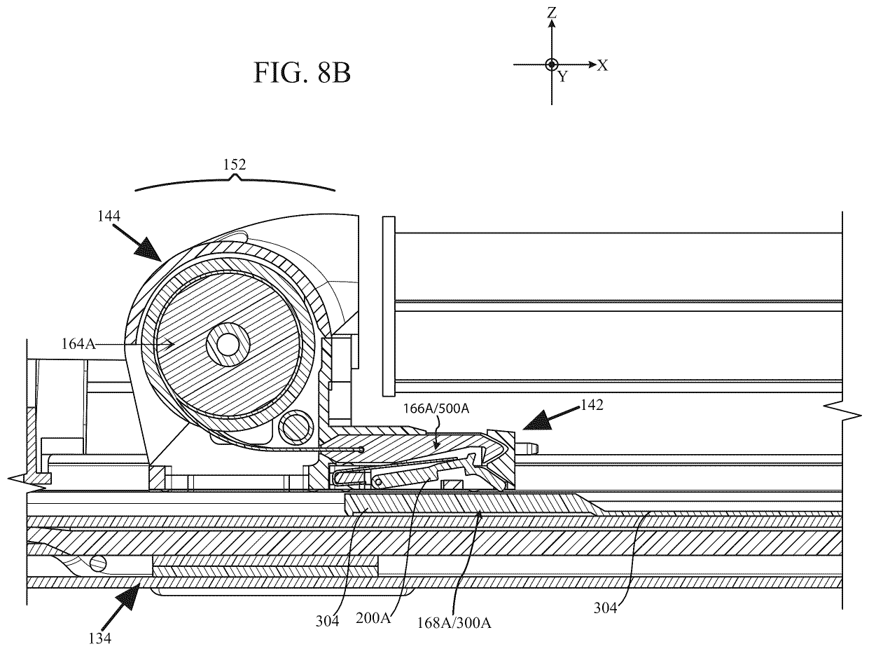

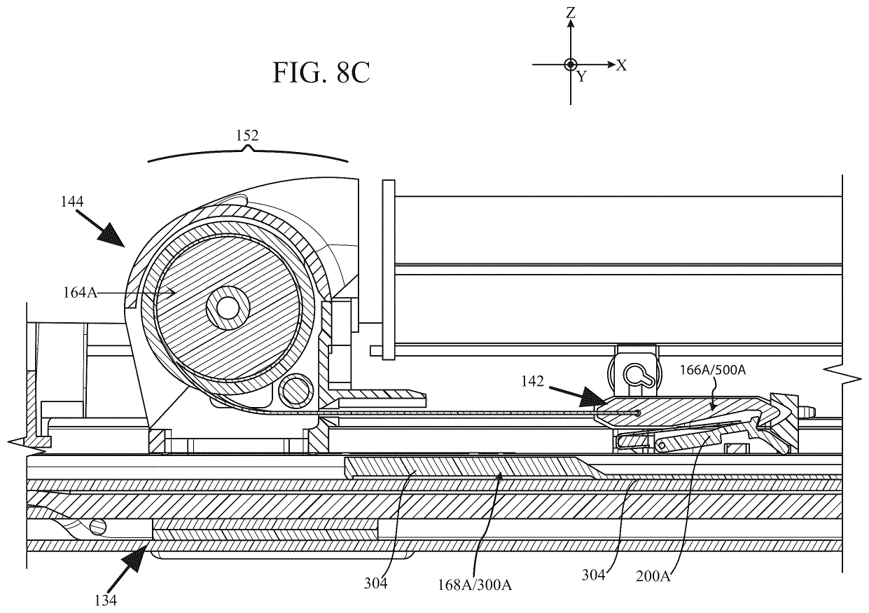

Continuing with the embodiment of CFPDSS 100 incorporating control rods 168 as tapered rods 300 having low sections 302 and high sections 304 as well as tapered pins 500 as the first ends 166 of tape springs 164, FIGS. 8A-8C presents a sequence of enlarged partial side views of the base 134 showing the puller 142, the pusher 144, a control rod 168/300 as well the engager 200 disposed below the first end 166/500 of a tape spring 164 for selective attachment to the puller 142.

As shown in FIG. 8A, CFPDSS 100 is essentially at rest as no syringe has yet been disposed and control rod 168A/300A is in its initial position with the low section 302 beneath engager 200. In FIG. 8B, a syringe has now been disposed within CFPDSS 100 such that the selector 150 (not shown) has been disposed towards the distal end 130 (not shown), which has resulted in the longitudinal movement of control rod 168A/300A towards distal end 130. As such the high section 304 has now moved to be beneath engager 200A, and consequently engager 200A has been raised up for engagement with first end 166A/500A.

In FIG. 8C, the cover 136 (not shown) has been at least partially closed, so as to engage the linkage 156 (not shown) to move the puller 142 away from the pusher 144, drawing out tape spring 164A as the first end 166A/500A is engaged by engager 200A to the puller 142. In other words, closing the cover 136 (not shown) tensions the selectively engaged tape springs 164, as the puller is moved towards the distal end 130 of CFPDSS 100.

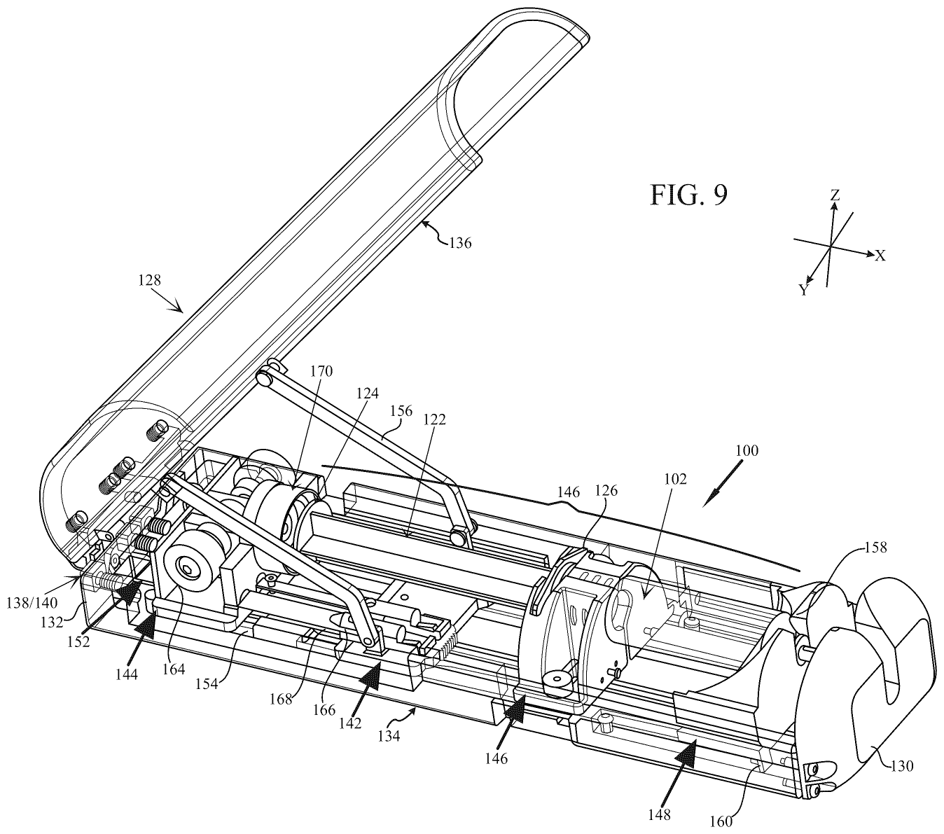

With respect to the above descriptions, FIG. 9 is a top perspective view of CFPDSS 100 with the cover now shown to be moving down to the closed position. As such, it may be appreciated that the linkage between the cover and the pusher 144 has advanced the pusher 144 away from the puller 142 and towards the distal end 130. It is this action that has been shown in the partial enlarged side views of FIGS. 8A-8C.

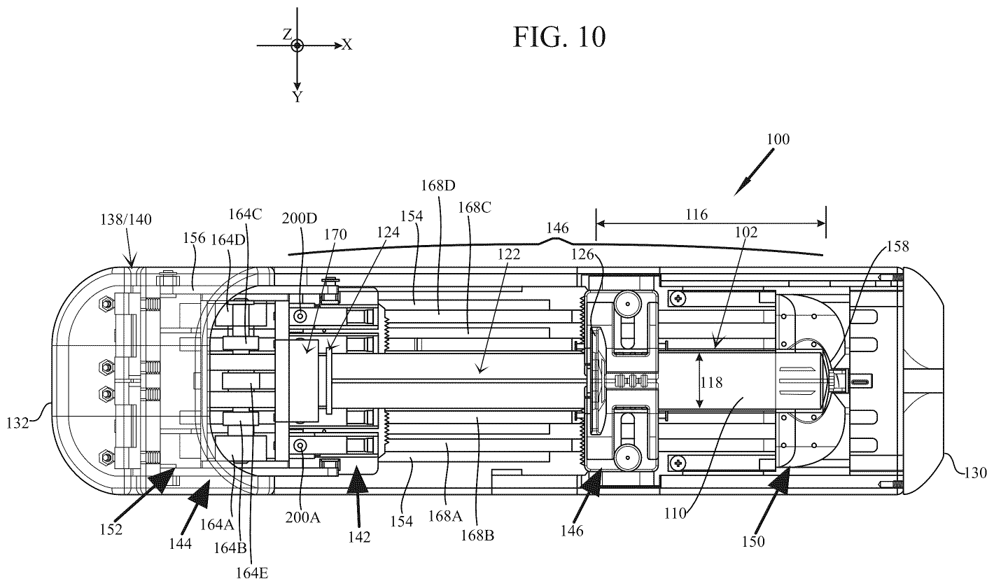

FIG. 10 is presented as a top view of CFPDSS 100 further permitting an appreciation of the above-described elements, such as, but not limited to the puller 142, the pusher 144, the collar 146, the sizer 148, the selector 150 and the adjustable driver 152.

With respect to the above description, it may be appreciated that at least one embodiment of a CFPDSS 100 accommodating syringes 102 of different sizes may be summarized as: a base 134 having a proximal end 132 and a distal end 130, the base 134 structured and arranged to receive a syringe 102 having a plunger 122 slidably disposed within a chamber 110 having an outlet 108, the chamber 110 having a length 116 and a diameter 114, the plunger 122 having a head 124; a pusher 144 in sliding engagement with the base 134, the pusher 144 structured and arranged to contact the head 124 of the plunger 122; a puller 142 in sliding engagement with the base 134; a sizer 148 structured and arranged to determine the size of the chamber 110; an adjustable driver 152 structured and arranged to provide a constant force between the pusher 144 and the puller 142, the constant force adjustably selected by the size of the chamber 110; whereby slidably moving the puller 142 distally when the syringe 102 is seated in the base 134 causes the pusher 144 to contact and exert a substantially constant force specific for the size of the chamber 110 on the head 124 of the plunger 122, the constant force sufficient to move the plunger 122 of the syringe 102; and a cover 136 connected to the base 134, a linkage 156 coupled between the cover 136 and the puller 142 and structured and arranged to translate the opening of the cover 136 to the movement of the puller 142 and pusher 144 towards the proximal end 132, and the closing of the cover 136 when the syringe 102 has been disposed engages the pusher 144 against the plunger 122 and moves the puller 142 towards the distal end 130 and engages the adjustable driver 152.

Yet another embodiment of a CFPDSS 100 may be summarized as: an expandable base 134 having a proximal end 132 and a distal end 130, the base 134 including: a first base 134 section and a second base 134 section, wherein the first base 134 section is in sliding engagement with the second base 134 section such that the first base 134 section and the second base 134 section are slidably movable relative to each other between a compacted position and an expanded position, wherein the base 134 in the expanded position is adapted to seat a syringe 102 having a plunger 122 slidably disposed within a chamber 110 having an outlet 108, the chamber 110 having a length 116 and a diameter 114, the plunger 122 having a head 124; a pusher 144 in sliding engagement with the base 134, wherein the pusher 144 is dimensioned to contact the head 124 of the plunger 122; a puller 142 in sliding engagement with the base 134; a sizer 148 structured and arranged to determine the size of the chamber 110; an adjustable driver 152 structured and arranged to provide a constant force between the pusher 144 and the puller 142, the constant force adjustably selected by the size of the chamber 110; whereby slidably moving the puller 142 distally when the syringe 102 is seated in the base 134 causes the pusher 144 to contact and exert a substantially constant force specific for the size of the chamber 110 on the head 124 of the plunger 122, the constant force sufficient to move the plunger 122 of the syringe 102; and an expandable cover 136 pivotally connected to the proximal end 132 of the base 134, a linkage 156 pivotally coupled between the cover 136 and the puller 142 and structured and arranged to translate the opening of the cover 136 to the movement of the puller 142 and pusher 144 towards the proximal end 132, and the closing of the cover 136 when the syringe 102 has been disposed engages the pusher 144 against the plunger 122 and moves the puller 142 towards the distal end 130 and engages the adjustable driver 152.

And further still, yet another embodiment of a CFPDSS 100 may be summarized as: a base 134 having a proximal end 132 and a distal end 130, the base 134 structured and arranged to receive a syringe 102 having a plunger 122 slidably disposed within a chamber 110 having an outlet 108, the chamber 110 having a length 116 and a diameter 114, the plunger 122 having a head 124; a pusher 144 in sliding engagement with the base 134, the pusher 144 structured and arranged to contact the head 124 of the plunger 122; a puller 142 in sliding engagement with the base 134; a sizer 148 structured and arranged to determine the size of the chamber 110; a plurality of different springs, each spring having a first end and a second end, the first end of each spring connected to the pusher 144, the second end of each spring selectively attached to the puller 142, the selection of at least one selectively attached spring from the plurality of springs base 134 on the size of the chamber 110; whereby slidably moving the puller 142 distally when the syringe 102 is seated in the base 134 causes the pusher 144 to contact and exert a substantially constant force specific for the size of the chamber 110 on the head 124 of the plunger 122, the constant force sufficient to move the plunger 122 of the syringe 102; and a cover 136 pivotally connected to the proximal end 132 of the base 134, a linkage 156 pivotally coupled between the cover 136 and the puller 142 and structured and arranged to translate the opening of the cover 136 to the movement of the puller 142 and pusher 144 towards the proximal end 132, and the closing of the cover 136 when the syringe 102 has been disposed engages the pusher 144 against the plunger 122 and moves the puller 142 towards the distal end 130 and tensions the at least one selectively attached spring; wherein the base 134 and cover 136 enclose the chamber 110 body, the at least one selectively attached spring, puller 142 and pusher 144 are structured and arranged to provide the constant force specific to the size of the chamber 110 to the plunger 122 of the syringe 102 providing a pressure pump.

Having described embodiments of the CFPDSS 100, other embodiments relating to at least one method of using CFPDSS 100 for providing infusion therapy into subcutaneous tissues will now be discussed. It will be appreciated that the described methods need not be performed in the order in which they are herein described, but that these descriptions are merely exemplary of methods for providing and using CFPDSS 100.

KORU Medical Systems, Inc. of Mahwah, New Jersey, is and has been a pioneer in infusion pump technology, needle set technology and flow rate control by means of specifically engineered flow control tubing. Indeed, KORU has realized that different flow rates may be provided by working with different flow combinations of flow control tubing, such as those systems and methods set forth in U.S. Pat. No. 10,420,886 entitled MULTI-FLOW UNIVERSAL TUBING SET, incorporated herein by reference, and U.S. Pat. No. 10,709,839 entitled PRECISION VARIABLE FLOW RATE INFUSION SYSTEM AND METHOD, incorporated herein by reference.

Further, KORU has developed advantageous infusion systems permitting high flow at low pressure as set forth in U.S. application Ser. No. 17/729,914 published as US 2022/0265923 entitled HIGH FLOW AT LOW PRESSURE INFUSION SYSTEM, incorporated herein by reference. Further still, for at least one embodiment the needle hub 100 is the snap in needle structure shown and described as element 162 in U.S. patent application Ser. No. 18/216,342 entitled SYSTEM AND METHOD FOR BUTTERFLY NEEDLE ASSEMBLY, incorporated herein by reference.

Moreover, it will be understood and appreciated that CFPDSS 100 may serve as the constant force pump for infusion therapy provided to a patient incorporating one or more of the above identified technologies. In addition, as noted above, for varying embodiments, where the syringe is coupled to a lure, such as the flared luer as set forth in U.S. Pat. No. 10,500,389 entitled, SYSTEM AND METHOD FOR FLARED LUER CONNECTOR FOR MEDICAL TUBING or a tapered luer as set forth in U.S. Provisional Application 63/616,368 entitled, SYSTEM AND METHOD FOR A TAPERED LURE CONNECTOR FOR MEDICAL TUBING, the selector 150, and more specifically the seat 158 of the selector may be structured and arranged as an accepting base for such a tapered or flared luer.

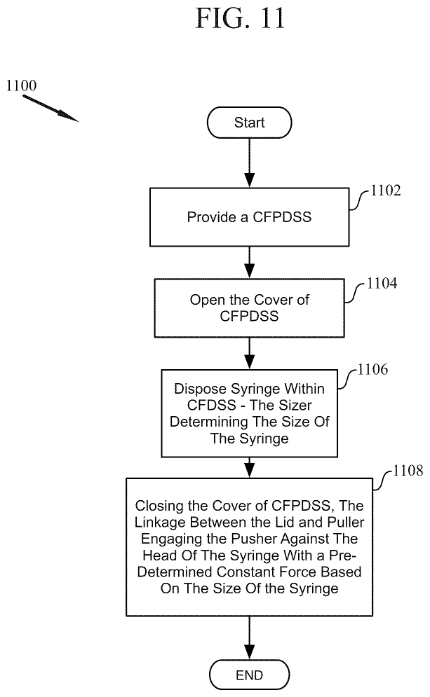

Turning to FIG. 11, presented is a flow diagram for at least one method 1100 of using CFPDSS 100 for infusion therapy. In general, method 1100 commences with providing a CFPDSS 100, block 1102.

The cover 136 of CFPDSS 100 it then opened, block 1104. A selected syringe is then disposed within CFPDSS 100. As discussed above, CFPDSS 100 is structured and arranged to use a sizer 148 to determine the size of the disposed syringe 102 and mechanically engage and adjustable driver 152 to provide a pre-determined constant force appropriate for the disposed syringe, block 1106.

To fully engage CFPDSS 100 with the pre-determined constant force, the cover 136 of CFPDSS 100 is then closed, and the infusion process is initiated to the patient, block 1108. As CFPDSS 100 is advantageously structured and arranged to determine the size of the syringe, and then the pre-determined constant force for the syringe based on the determined size, it will be understood and appreciated that the same CFPDSS 100 may be used for the infusion therapy treatment of different sized syringes without adverse effects from improper force applied for the administration. Moreover, a single CFPDSS 100 is advantageously capable of self-determining different syringe sizes and automatically selecting the pre-determined constant force appropriate for the administration of the medicate within a syringe of a determined size.

Moreover, with respect to the above method description, it may be appreciated that at least one embodiment of a method for using a CFPDSS 100 may be summarized as: providing a pump enclosure including: a base 134 having a proximal end 132 and a distal end 130, the base 134 structured and arranged to receive a syringe 102 having a plunger 122 slidably disposed within a chamber 110 having an outlet 108, the chamber 110 having a length 116 and a diameter 114, the plunger 122 having a head 124; a pusher 144 in sliding engagement with the base 134, the pusher 144 structured and arranged to contact the head 124 of the plunger 122; a puller 142 in sliding engagement with the base 134, the puller 142 structured and arranged to receive a syringe 102 having a plunger 122 set to extrude a liquid from a chamber 110, the chamber 110 having a length 116 and a diameter 114; a sizer 148 structured and arranged to determine the size of the chamber 110; an adjustable driver 152 structured and arranged to provide a constant force between the pusher 144 and the puller 142, the constant force adjustably selected by the size of the chamber 110; whereby slidably moving the puller 142 distally when the syringe 102 is seated in the base 134 causes the pusher 144 to contact and exert a substantially constant force specific for the size of the chamber 110 on the head 124 of the plunger 122, the constant force sufficient to move the plunger 122 of the syringe 102; and an expandable cover 136 connected to the proximal end 132 of the base 134, a linkage 156 coupled between the cover 136 and the puller 142 and structured and arranged to translate the opening of the cover 136 to the movement of the puller 142 and pusher 144 towards the proximal end 132, and the closing of the cover 136 when the syringe 102 has been disposed engages the pusher 144 against the plunger 122 and moves the puller 142 towards the distal end 130 and engages the adjustable driver 152; wherein the expandable base 134 and expandable cover 136 enclose the chamber 110 body, the adjustable driver 152, puller 142 and pusher 144 are structured and arranged to provide force specific to the length 116 of the chamber 110 to the plunger 122 of the syringe 102 providing a pressure pump; opening the expandable cover 136 of the pump enclosure; seating a selected syringe 102 within the expandable base 134, the sizer 148 determining a size of the selected syringe 102, the determined size selectively engaging adjustable driver 152; and closing the expandable cover 136 to engage the pusher 144 against the plunger 122 of the selected syringe 102.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A constant force syringe pump assembly accommodating syringes of different sizes comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base;
a sizer structured and arranged to determine the size of the chamber;
an adjustable driver structured and arranged to provide a constant force between the pusher and the puller, the constant force adjustably selected by the size of the chamber;
whereby slidably moving the puller distally when the syringe is seated in the base causes the pusher to contact and exert a substantially constant force specific for the size of the chamber on the head of the plunger, the constant force sufficient to move the plunger of the syringe; and
a cover connected to the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

2. The constant force syringe pump of claim 1, wherein the adjustable driver includes a plurality of different springs, each spring having a first end and a second end, the first end of each spring connected to the pusher, the second end of each spring selectively attached to the puller, the selection of at least one selectively attached spring from the plurality of springs based on the size of the chamber.

3. The constant force syringe pump of claim 2, wherein the selection of selectively attached springs ensures a pre-determined constant force appropriate to the size of the chamber is exerted upon the syringe disposed within the pump enclosure assembly.

4. The constant force syringe pump of claim 2, wherein the pre-determined constant force appropriate to the size of the chamber ensures an outflow rate from the syringe chamber does not exceed a pre-determined rate.

5. The constant force syringe pump of claim 2, wherein there at least two different springs.

6. The constant force syringe pump of claim 1, wherein the cover is pivotally connected to the proximal end of the base, the linkage pivotally coupling the cover and the puller.

7. The constant force syringe pump of claim 1, wherein the size of the chamber is determined by the length.

8. The constant force syringe pump of claim 1, wherein the size of the chamber is determined by the diameter.

9. The constant force syringe pump of claim 1, wherein the adjustable constant force selected is between about 1 to 25 pounds of force.

10. The constant force syringe pump of claim 1, wherein the pump is structured and arranged to accommodate a syringe having a volume of at least 5 milliliters and no more than 120 milliliters.

11. The constant force syringe pump of claim 1, wherein the sizer is a mechanical system comprising:
a plurality of levers, each lever corresponding to a different spring; and
a selector, the selector being movable between a first position to a second position established by the size of the chamber, the sliding of the selector from the first position to the second position causing one or more of the levers to engage one or more of the springs, thereby selectively attaching one or more springs to the puller.

12. The constant force syringe pump of claim 1, wherein the base and the cover are expandable.

13. A constant force syringe pump assembly accommodating syringes of different sizes comprising:
an expandable base having a proximal end and a distal end, the base comprising: a first base section and a second base section, wherein the first base section is in sliding engagement with the second base section such that the first base section and the second base section are slidably movable relative to each other between a compacted position and an expanded position, wherein the base in the expanded position is adapted to seat a syringe having a plunger slidably disposed within a chamber having an outlet, the chamber having a length and a diameter, the plunger having a head;
a pusher in sliding engagement with the base, wherein the pusher is dimensioned to contact the head of the plunger;
a puller in sliding engagement with the base;
a sizer structured and arranged to determine the size of the chamber;
an adjustable driver structured and arranged to provide a constant force between the pusher and the puller, the constant force adjustably selected by the size of the chamber;
whereby slidably moving the puller distally when the syringe is seated in the base causes the pusher to contact and exert a substantially constant force specific for the size of the chamber on the head of the plunger, the constant force sufficient to move the plunger of the syringe; and
an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

14. The constant force syringe pump of claim 13, wherein the adjustable driver includes a plurality of different springs, each spring having a first end and a second end, the first end of each spring connected to the pusher, the second end of each spring selectively attached to the puller, the selection of at least one selectively attached spring from the plurality of springs based on the size of the chamber.

15. The constant force syringe pump of claim 14, wherein the selection of selectively attached springs ensures a pre-determined constant force appropriate to the size of the chamber is exerted upon the syringe disposed within the pump enclosure assembly.

16. The constant force syringe pump of claim 14, wherein the pre-determined constant force appropriate to the size of the chamber ensures an outflow rate from the syringe chamber does not exceed a pre-determined rate.

17. The constant force syringe pump of claim 14, wherein there at least two different springs.

18. The constant force syringe pump of claim 13, wherein the cover is pivotally connected to the proximal end of the base, the linkage pivotally coupling the cover and the puller.

19. The constant force syringe pump of claim 13, wherein the cover is slidably connected to the base, the linkage slidably coupling the cover and the puller.

20. The constant force syringe pump of claim 13, wherein the size of the chamber is determined by the length.

21. The constant force syringe pump of claim 13, wherein the size of the chamber is determined by the diameter.

22. The constant force syringe pump of claim 13, wherein the adjustable constant force selected is between about 1 to 25 pounds of force.

23. The constant force syringe pump of claim 13, wherein the pump is structured and arranged to accommodate a syringe having a volume of at least 5 milliliters and no more than 120 milliliters.

24. The constant force syringe pump of claim 13, wherein the sizer is a mechanical system comprising:
a plurality of levers, each lever corresponding to a different spring; and
a selector, the selector being movable between a first position to a second position established by the size of the chamber, the sliding of the selector from the first position to the second position causing one or more of the levers to engage one or more of the springs, thereby selectively attaching one or more springs to the puller.

25. The constant force syringe pump of claim 13, wherein the base and the cover are expandable.

26. A constant force syringe pump assembly accommodating syringes of different sizes comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base;
a sizer structured and arranged to determine the size of the chamber;
a plurality of different springs, each spring having a first end and a second end, the first end of each spring connected to the pusher, the second end of each spring selectively attached to the puller, the selection of at least one selectively attached spring from the plurality of springs based on the size of the chamber;
whereby slidably moving the puller distally when the syringe is seated in the base causes the pusher to contact and exert a substantially constant force specific for the size of the chamber on the head of the plunger, the constant force sufficient to move the plunger of the syringe; and
a cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one selectively attached spring;
wherein the base and cover enclose the chamber body, the at least one selectively attached spring, puller and pusher are structured and arranged to provide the constant force specific to the size of the chamber to the plunger of the syringe providing a pressure pump.

27. The constant force syringe pump of claim 26, wherein the selection of selectively attached springs ensures a pre-determined constant force appropriate to the size of the chamber is exerted upon the syringe disposed within the pump enclosure assembly.

28. The constant force syringe pump of claim 27, wherein the pre-determined constant force appropriate to the size of the chamber ensures an outflow rate from the syringe chamber does not exceed a pre-determined rate.

29. The constant force syringe pump of claim 26, wherein the size of the chamber is determined by the length.

30. The constant force syringe pump of claim 26, wherein the size of the chamber is determined by the diameter.

31. The constant force syringe pump of claim 26, wherein the adjustable constant force selected is between about 1 to 25 pounds of force.

32. The constant force syringe pump of claim 26, wherein the pump is structured and arrange to accommodate a syringe having a volume of at least 5 milliliters and no more than 120 milliliters.

33. The constant force syringe pump of claim 26, wherein there at least two different springs.

34. The constant force syringe pump of claim 26, wherein the sizer is a mechanical system comprising:
a plurality of levers, each lever corresponding to a different spring; and
a selector, the selector being movable between a first position to a second position established by the size of the chamber, the sliding of the selector from the first position to the second position causing one or more of the levers to engage one or more of the springs, thereby selectively attaching one or more springs to the Puller.

35. The constant force syringe pump of claim 26, wherein the base and the cover are expandable.

36. A method for using a constant force syringe pump enclosure assembly accommodating syringes of different sizes to dispense a solution from a syringe, comprising:
providing a pump enclosure comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base, the puller structured and arranged to receive a syringe having a plunger set to extrude a liquid from a chamber, the chamber having a length and a diameter;
a sizer structured and arranged to determine the size of the chamber;
an adjustable driver structured and arranged to provide a constant force between the pusher and the puller, the constant force adjustably selected by the size of the chamber;
whereby slidably moving the puller distally when the syringe is seated in the base causes the pusher to contact and exert a substantially constant force specific for the size of the chamber on the head of the plunger, the constant force sufficient to move the plunger of the syringe; and
an expandable cover connected to the proximal end of the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver;
wherein the expandable base and expandable cover enclose the chamber body, the adjustable driver, puller and pusher are structured and arranged to provide force specific to the length of the chamber to the plunger of the syringe providing a pressure pump;
opening the expandable cover of the pump enclosure;
seating a selected syringe within the expandable base, the sizer determining a size of the selected syringe, the determined size selectively engaging adjustable driver; and
closing the expandable cover to engage the pusher against the plunger of the selected syringe.

37. The method claim 36, wherein the adjustable driver includes a plurality of different springs, each spring having a first end and a second end, the first end of each spring connected to the pusher, the second end of each spring selectively attached to the puller, the selection of at least one selectively attached spring from the plurality of springs based on the size of the chamber.

38. The method of claim 37, wherein the selection of selectively attached springs ensures a pre-determined constant force appropriate to the size of the chamber is exerted upon the syringe disposed within the pump enclosure assembly.

39. The method of claim 37, wherein the pre-determined constant force appropriate to the size of the chamber ensures an outflow rate from the syringe chamber does not exceed a pre-determined rate.

40. The method of claim 37, wherein there at least two different springs.

41. The method of claim 36, wherein the cover is pivotally connected to the proximal end of the base, the linkage pivotally coupling the cover and the puller.

42. The method of claim 36, wherein the cover is slidably connected to the base, the linkage slidably coupling the cover and the puller.

43. The method of claim 36, wherein the size of the chamber is determined by the length.

44. The method of claim 36, wherein the size of the chamber is determined by the diameter.

45. The method of claim 36, wherein the adjustable constant force selected is between about 1 to 25 pounds of force.

46. The method of claim 36, wherein the pump is structured and arranged to accommodate a syringe having a volume of at least 5 milliliters and no more than 120 milliliters.

47. The method of claim 36, wherein the sizer is a mechanical system comprising:
a plurality of levers, each lever corresponding to a different spring; and
a selector, the selector being movable between a first position to a second position established by the size of the chamber, the sliding of the selector from the first position to the second position causing one or more of the levers to engage one or more of the springs, thereby selectively attaching one or more springs to the Puller.

48. The method of claim 36, wherein the base and the cover are expandable.

49. A method for using a constant force syringe pump enclosure assembly accommodating syringes of different sizes to dispense a solution from a syringe, comprising:
providing a pump enclosure comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base, the puller structured and arranged to receive a syringe having a plunger set to extrude a liquid from a chamber, the chamber having a length and a diameter;
a sizer structured and arranged to determine the size of the chamber;
a plurality of different springs, each spring having a first end and a second end, the first end of each spring connected to the pusher, the second end of each spring selectively attached to the puller, the selection of at least one selectively attached spring from the plurality of springs based on the length of the chamber;
whereby slidably moving the puller distally when the syringe is seated in the base causes the pusher to contact and exert a substantially constant force specific for the size of the chamber on the head of the plunger, the constant force sufficient to move the plunger of the syringe; and
an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one selectively attached spring;
wherein the expandable base and expandable cover enclose the chamber body, the at least one selectively attached spring, puller and pusher are structured and arranged to provide force specific to the length of the chamber to the plunger of the syringe providing a pressure pump;
opening the expandable cover of the pump enclosure;
seating a selected syringe within the expandable base, the sizer determining a size of the selected syringe, the determined size selectively engaging at least one spring from the plurality of springs; and
closing the expandable cover to engage the pusher against the plunger of the selected syringe.

50. The method of claim 49, wherein the selection of selectively attached springs ensures a pre-determined constant force appropriate to the size of the chamber is exerted upon the syringe disposed within the pump enclosure assembly.

51. The method of claim 50, wherein the pre-determined constant force appropriate to the size of the chamber ensures an outflow rate from the syringe chamber does not exceed a pre-determined rate.

52. The method of claim 49, wherein the size of the chamber is determined by the length.

53. The method of claim 49, wherein the size of the chamber is determined by the diameter.

54. The method of claim 49, wherein the adjustable constant force selected is between about 1 to 25 pounds of force.

55. The method of claim 49, wherein the pump is structured and arranged to accommodate a syringe having a volume of at least 5 milliliters and no more than 120 milliliters.

56. The method of claim 49, wherein there at least two different springs.

57. The method of claim 49, wherein the sizer is a mechanical system comprising:
a plurality of levers, each lever corresponding to a different spring; and
a selector, the selector being movable between a first position to a second position established by the size of the chamber, the sliding of the selector from the first position to the second position causing one or more of the levers to engage one or more of the springs, thereby selectively attaching one or more springs to the puller.
